(19) United States
(12) Patent Application Publication
(10) Pub. No.: US 2018/0099024 A1
(43) Pub. Date: Apr. 12, 2018

(54) METHODS, COMPOSITIONS, AND KITS INVOLVING ALTERATION OF THE CHAPERONE PROTEIN AXIS IN A SUBJECT

(71) Applicant: RAMP RESEARCH, LLC, The Woodlands, TX (US)

(72) Inventor: Patrick J. Casey, Kumeu (NZ)

(21) Appl. No.: 15/726,224

(22) Filed: Oct. 5, 2017

Related U.S. Application Data
(60) Provisional application No. 62/405,005, filed on Oct. 6, 2016.

(51) Int. Cl. A61K 38/17; A61K 35/57; A61K 9/00; A61P 21/00; A61P 43/00; A61P 3/08; A61P 37/00; A61P 35/00; A61P 13/12; A61P 25/28; A61P 15/00; A61P 19/00; A61P 25/16; A61P 5/24; G01N 33/68

(52) U.S. Cl. CPC A61K 38/1709; G01N 2800/52; A61K 9/0019; A61P 21/00; A61P 43/00; A61P 3/08; A61P 37/00; A61P 35/00; A61P 13/12; A61P 25/28; A61P 15/00; A61P 19/00; A61P 25/16; A61P 5/24; G01N 33/68; A61K 35/57

(57) ABSTRACT

The present invention broadly related to compositions, treatment methods, and kits involving alteration of the chaperone protein axis of a subject. In one aspect, the invention features a method of treatment of a subject including the step of administering to the subject at least one dose of a therapeutically effective amount of a composition for altering a chaperone protein axis of the subject. The composition includes at least one essential fragment of a feto-placental unit protein.

### STATEMENT REGARDING FEDERALLY SPONSORED RESEARCH OR DEVELOPMENT

Not Applicable

### BACKGROUND OF THE INVENTION

The present invention broadly relates to compositions, treatment methods, and kits involving alteration of the chaperone protein axis or CPA in a subject.

Research has linked chaperone proteins, and in particular, the 70-kDa heat shock protein (HSP70) family of chaperone proteins, with cellular proteostasis including folding, transport, and assembly of proteins within, through and outside the cell. Jurgen Radons. Cell Stress and Chaperones (2016) 21:3709-404 (hereinafter “Radons 2016”). HSP70 proteins are found within the cell, on the cell surface and extracellularly. Id. Intracellular HSP70s have been associated with protection of cells against stress. Radons 2016 citing Hartl FU (1996) Molecular chaperones in cellular protein folding. Nature 381:571-579. Extracellular HSP70s have been associated with the modification of the immune response or functioning of the immune system. Radons 2016 citing Henderson B, Pockley A G (2010) Molecular chaperones and protein folding catalysts as intercellular signaling regulators in immunity and inflammation. J Leukoc Biol 88:445-462 among others.

Changes in intra-cellular and extra-cellular HSP70 levels have been associated with diseased versus healthy states. Radons 2016. For example, higher levels of intra-cellular HSP70 proteins in cancerous cells relative to non-malignant cells have been observed. Radons 2016. Higher levels of extracellular HSP70 proteins in circulating serum have been associated with inflammation and frailty in the elderly. Radons 2016 citing Njemini R, Bautmans I, Onyema O O et al (2011) Circulating heat shock protein 70 in health, aging and disease. BMC Immunol 12:24. It has also been suggested that circulating serum HSP70 may indicate inflammation in healthy individuals. Radons 2016. There is some evidence that the concentration of extracellular circulating HSP70 in the blood stream decreases with age in healthy individuals. Id. It has been proposed that low levels of circulating serum HSP70 may “serve as an indicator of a healthy state and a biomarker for longevity”. Radons 2016 citing Terry D F, McCormick M, Andersen S et al (2004) Cardiovascular disease delay in centenarian offspring: role of heat shock proteins. Ann N Y Acad Sci 1019:502-505.

There is a need to find ways to exploit both the activity and the possible modification of chaperone proteins in medical and veterinary care, including prevention and treatment following disease, treatment or other trauma.

### BRIEF SUMMARY OF THE INVENTION

The invention features methods, compositions and kits related to the alteration of the chaperone protein axis in a subject.

In one aspect, the invention features a method of treatment of a subject including the step of administering to the subject at least one dose of a therapeutically effective amount of a composition for altering a chaperone protein axis of the subject. The composition includes at least one essential fragment of a feto-placental unit protein. In a preferred embodiment, the feto-placental unit protein is a mammalian feto-placental unit protein.

In one embodiment, the essential fragment of the feto-placental unit protein includes the essential fragment of a chaperone protein. In a preferred embodiment, the chaperone protein includes a protein included in the HSP70 family.

In another embodiment, the method of treatment includes prior to the administering step, identifying a subject in need of alteration of the chaperone protein axis of the subject.

In other embodiments of the treatment methods of the invention, altering the chaperone protein axis of the subject includes stimulating the chaperone protein axis for at least one of manufacturing a chaperone protein, releasing a chaperone protein from a cell, decreasing an amount of a chaperone protein, and increasing an amount of a chaperone protein in the subject. In a preferred embodiment, altering the chaperone protein axis of the subject includes decreasing an amount of a chaperone protein circulating in a blood stream of the subject.

In still other embodiments, the treatment method of the invention includes monitoring the chaperone protein axis in the subject during at least one of a selected pre-treatment time period, and treatment time period, and a post-treatment time period. In a preferred embodiment, the monitoring includes measuring at least one of an intracellular amount and an extracellular amount of a key protein which is involved in the chaperone protein axis in the subject. In a more preferred embodiment, the key protein includes a protein included in the HSP70 family.

In yet other embodiments of the treatment method of the invention, altering the chaperone protein axis of the subject includes at least one of growing, regenerating, and repairing a cell in the subject.

In a preferred embodiment of the treatment method of the invention, the therapeutically effective amount is in a range of 3 mg of the composition per 100 kg body weight of the subject to 3 g of the composition per 100 kg body weight of the subject.

In different embodiments, the inventive treatment method includes administering the at least one dose of the therapeutically effect amount of the composition using at least one of a plurality of procedures selected from the group consisting of an oral administration, a rectal administration, an inhalation administration, a cutaneous administration, a subcutaneous administration, a transcutaneous administration, an intravenous injection, and an intramuscular injection.

In a preferred embodiment, the inventive treatment method includes administering the at least one dose of the therapeutically effective amount of the composition in combination with at least one additional component for achieving an additive or synergistic effect. In a more preferred embodiment, the inventive treatment method includes administering using a transcutaneous administration the at least one dose of the therapeutically effective amount of the composition in combination with at least one additional component for achieving an additive or synergistic effect. In a preferred embodiment, the additional component includes an oil, and in a more preferred embodiment, an emu oil.

In another embodiment, the inventive treatment method includes administering to the subject the at least one dose at a selected dosage rate during a selected dosing time interval. In one embodiment, the selected dosage rate is in a range of one dose per one day to one dose per 365 days during a selected dosing time interval. In another embodiment, the inventive treatment method includes a selected dosing time interval in a range of a single day to a day substantially proximate to an expected end of life of the subject.

In another embodiment, the inventive treatment method includes altering the chaperone protein axis in the subject including altering a level of the chaperone protein axis from a pre-alteration chaperone protein axis level to a post-alteration chaperone protein axis level which is maintained for a sustaining period of time. In one embodiment, the sustaining period of time is in a range of 1 week to 52 weeks. In another embodiment, the post-alteration chaperone protein axis level is maintained for a sustaining period of time after the at least one dose is no longer being administered to the subject.

In still another embodiment of the inventive treatment method, the subject is a mammal. In preferred embodiments of the inventive treatment method, the mammal is selected from the group of genus species classification consisting of Equus caballus and Homo sapiens. In a more preferred embodiment, the subject is a human. In another preferred embodiment, the subject is a horse.

In another aspect, the invention features a pharmaceutical composition including a therapeutically effective amount of at least an essential fragment of a feto-placental unit protein for altering a chaperone protein axis in a subject. In a preferred embodiment, the feto-placental unit protein is a mammalian feto-placental unit protein.

In an embodiment, the invention features a kit containing the pharmaceutical composition of the invention.

In preferred embodiments, in a subject which is a human, the therapeutically effective amount is in a range from about 10 ug feto-placental unit protein/kg body weight to 1 mg feto-placental unit protein/kg body weight in the subject; and in the subject which is a horse, therapeutically effective amount is in a range from about 1 ug feto-placental unit protein/kg body weight to 1 mg feto-placental unit protein/kg body weight in the subject.

In still another aspect, the invention features a method of assessing an ability of a subject to recover from a condition related to a defect or a disease, injury or other trauma. The method includes the steps of: administering at least one dose of an effective amount of a composition to a subject for altering a level of a chaperone protein axis in the subject; wherein the composition includes at least an essential fragment of a feto-placental unit protein; measuring at least one pre-alteration amount of at least one key protein of the chaperone protein axis at at least one selected time interval before administering the at least one dose; measuring at least one post-alteration amount of the at least one key protein of the chaperone protein axis at at least one selected time interval following administering the at least one dose; and correlating with a first algorithm the difference in the at least one pre-alteration amount and the at least one post-alteration amount of the at least one key protein with an ability of the subject to recover from the condition. In a preferred embodiment, the feto-placental unit protein is a mammalian feto-placental unit protein.

In one embodiment, the condition is selected from the group consisting of a tendon injury, a muscle injury and at least one aging condition.

In another embodiment of the inventive assessment method, the essential fragment of the feto-placental unit protein includes an essential fragment of a chaperone protein. In a preferred embodiment, the chaperone protein comprises a protein included in the HSP70 family.

In a further embodiment of the inventive assessment method, the effective amount is in a range of 1 ug of the composition to 10 g of the composition per 50 kg body weight of the subject.

In still further embodiments of the inventive assessment method, administering includes at least one of a plurality of administration procedures selected from an oral administration, a rectal administration, an inhalation administration, a cutaneous administration, a subcutaneous administration, a transcutaneous administration, an intravenous injection, and an intramuscular injection.

In another embodiment, the inventive assessment method includes administering the at least one dose of the effective amount of the composition in combination with at least one additional component for achieving an additive or synergistic effect. In a preferred embodiment, the inventive assessment method includes administering transcutaneously the at least one dose in combination with at least one additional component for achieving an additive or synergistic effect. In a preferred embodiment, the additional component is an oil, and in an even more preferred embodiment, the oil is an emu oil.

In another embodiment, the inventive assessment method includes administering the at least one dose at a selected dosage rate during a selected dosing time interval. In one embodiment, the selected dosage rate is in a range of one dose per one day to one dose per 365 days during a selected dosing time interval. In another embodiment, the selected dosing time interval is in a range of a single day to a day substantially proximate to an expected end of life of the subject.

In an embodiment of the inventive assessment method, the subject is a mammal. In preferred embodiments, the mammal is selected from the group of genus species classifications consisting of Equus caballus and Homo sapiens. In a more preferred embodiment, the subject is a human. In another more preferred embodiment, the subject is a horse.

In another aspect, the invention includes an assessment kit for assessing an ability of a subject to recover from a condition related to a disease, injury, or other trauma. The kit includes a composition for altering a chaperone protein axis of the subject; wherein the composition includes at least an essential fragment of a feto-placental unit protein; and a device for administering the composition. In a preferred embodiment, the feto-placental unit protein is a mammalian feto-placental unit protein.

In one embodiment, the assessment kit includes one or more devices for measuring at least one pre-alteration amount of at least one key protein of the chaperone protein axis at at least one selected time interval before administering the composition and at least one post-alteration amount of the at least one key protein of the chaperone protein axis at at least one selected time interval following administering the composition.

In another embodiment, the kit includes at least one device for correlating with a first algorithm the difference in the at least one pre-alteration amount and the at least one post-alteration amount of the at least one key protein with an ability of the subject to recover from the condition.

### BRIEF DESCRIPTION OF THE SEVERAL VIEWS OF THE DRAWINGS

Further features of the present invention will become apparent from the following description of embodiments thereof, given by way of example only, with reference to the accompanying drawings, in which:

FIG. 1 shows the steps of harvesting, processing and storing a plurality of proteins from a mammalian feto-placental unit, as described in the method of the prior art;

FIG. 2 shows the steps of harvesting, processing and storing a plurality of proteins from a mammalian feto-placental unit, as described in the method of the prior art;

FIG. 3 shows the steps of harvesting, processing and storing a plurality of proteins from a mammalian feto-placental unit, as described in the method of the prior art;

FIG. 4 shows the steps of harvesting, processing and storing a plurality of proteins from a mammalian feto-placental unit, as described in the method of the prior art;

FIG. 5 shows schematically the mis-folding and refolding of a protein, as described in the prior art;

FIG. 6 shows schematically the degradation of a protein by a chaperone protein, as described in the prior art;

FIG. 7 shows schematically cell apoptosis, as described in the prior art;

FIG. 8 shows the steps of the method for administration and accumulation reduction, as described in the prior art

FIG. 9 shows experimental results achieved with a non-limiting embodiment of the invention;

FIG. 10 shows experimental results achieved with a non-limiting embodiment of the invention; and

FIG. 11 shows experimental results achieved with a non-limiting embodiment of the invention;

FIG. 12 shows experimental results achieved with a non-limiting embodiment of the invention;

FIG. 13 is a graph showing a treatment timeline of male and female cats treated according to a non-limiting embodiment of the invention;

FIG. 14 is a graph showing the level of GFR over time for control, untreated male cats;

FIG. 15 is a graph showing the level of GFR over time for control, untreated female cats;

FIG. 16 is a graph showing the effect on GFR over time for male cats treated according to a non-limiting embodiment of the invention;

FIG. 17 is a graph showing the effect on GFR over time for female cats treated according to a non-limiting embodiment of the invention;

FIG. 18 is a graph showing the level of HSP70 over time for control, untreated male and female cats;

FIG. 19 is a graph showing the level of HSP70 over time in treated male and female cats according to a non-limiting embodiment of the invention;

FIG. 20 is a graph showing the level of cell free DNA or cfDNA over time in male and female cats treated according to a non-limiting embodiment of the invention;

FIG. 21 is a graph showing a relationship between GFR, cellular necrosis, apoptosis and anastasis in subjects;

FIG. 22 shows experimental results achieved with a non-limiting embodiment of the invention;

FIG. 23 shows experimental results achieved with a non-limiting embodiment of the invention;

FIG. 24 shows a method of treatment according to a non-limiting embodiment of the invention; and

FIG. 25 shows a method of assessing an ability of a subject to recover from a condition related to a defect, disease, injury or other trauma according to a non-limiting embodiment of the invention.

### DETAILED DESCRIPTION OF THE INVENTION

This application incorporates by reference the entire disclosures of U.S. patent application Ser. Nos. 14/330,818 and 14/551,612, and claims priority to U.S. Provisional Application No. 62/405,005; filed Oct. 6, 2016.

### Definitions

For purposes of this invention, the following terms are defined as set forth below:

The terms “alter, alteration of and altering the chaperone protein axis or CPA” in a subject refer to an increase, a decrease, a change and/or other modification of the chaperone protein axis of the subject.

The terms “chaperone protein axis or CPA” of a subject refer to the intracellular, intercellular, and/or extracellular activities and/or effects of a chaperone protein or chaperone proteins in the subject including but not limited to the activities and/or effects of a chaperone protein or chaperone proteins in the biological systems and/or circulating blood stream of the subject.

The term “essential fragment(s) of a protein” refers to the portion of the protein which is capable of at least one of the reversal of cell apoptosis, the repair of cells, the growth and/or regeneration of cells, and the regulation of protein folding, re-folding, transportation and degradation, through, for a non-limiting example, the Ubiquitin Proteasome Pathway (UPP).

The term “placental-cord fetal component(s)” refer to the fetal placenta with cotyledons and umbilical cord which are harvested or dissected from a fetal mass extracted from a donor animal.

The term “fetal placental unit” refers to the dissected placenta-cord fetal component plus the remaining dissected fetal mass.

The term “dissected fetal mass” refers to a fetal component or components including, for example, a liver component, a spleen component, a whole brain component, an ocular component, a gastro-intestinal component, a lung component, a heart component, a pancreas component, a kidney component, a female specific component which includes accessory sexual glands, and/or a male specific component which includes male penile tissue, testicular tissue and/or accessory sexual glands.

The term “aging condition” refers to fragility, inflammation, type 2 diabetes, type 1 diabetes, cardiomyopathy, prostate cancer, renal dysfunction, intra-cellular storage disease, extra-cellular storage disease, Alzheimer disease, Parkinson disease, sexual dysfunction including female dysfunction (e.g., fluid reduction) and male dysfunction (e.g., erectile dysfunction), hormonal imbalance including hormonal imbalance around the cessation of menses or menopause, musculoskeletal disorder, and/or neoplasia.

The term “evaluation factor(s) of a subject” refer to factors such as age, gender, amount of pre-existing condition related to a defect, disease, injury or other trauma, genetic predisposition, and/or environment of the subject.

The term “intra- and extra-cellular storage disease” refers to conditions where the body makes proteins but does not have the mechanism such as, for example, the enzymes, to break down the proteins, and the accumulation of such proteins becomes harmful for the subject.

### Invention

The present invention relates to the methods, compositions and kits for the alteration of the chaperone protein axis in a subject. The prior art offers different interpretations regarding the role of chaperone proteins. For example, some researchers suggest that chaperone proteins aid in cellular proteostasis including protein folding, transport and assembly of proteins within, through and outside the cell, Radons 2016, while other researchers associate higher levels of intracellular or extracellular chaperone proteins with disease or otherwise unhealthy states, Id. citing, respectively, Njemini et al. 2011 and Terry et al. 2004, as discussed above.

In the present invention, it has been found that the administration of a dose of a therapeutically effective amount of a composition of the invention can alter the chaperone protein axis of a subject. Such administration can alter the chaperone protein axis by stimulating intracellular, intercellular and/or extracellular activities of one or more chaperone proteins in the subject. Such stimulation of the chaperone axis can result in the manufacture of a chaperone protein, release of a chaperone protein from a cell, and/or an increase or a decrease in the intracellular, intercellular, or extracellular amount and/or activities of a chaperone protein.

The composition of the invention includes at least one essential fragment of a feto-placental unit protein. Preferably, the feto-placental unit protein is a chaperone protein and more preferably, the feto-placental unit protein is a protein included in the HSP70 family. The feto-placental unit can include tissue including the fetal placenta, intestinal tract, lungs, heart, liver, pancreas, and/or kidneys. In a preferred embodiment, the feto-placental unit protein is a mammalian feto-placental unit protein.

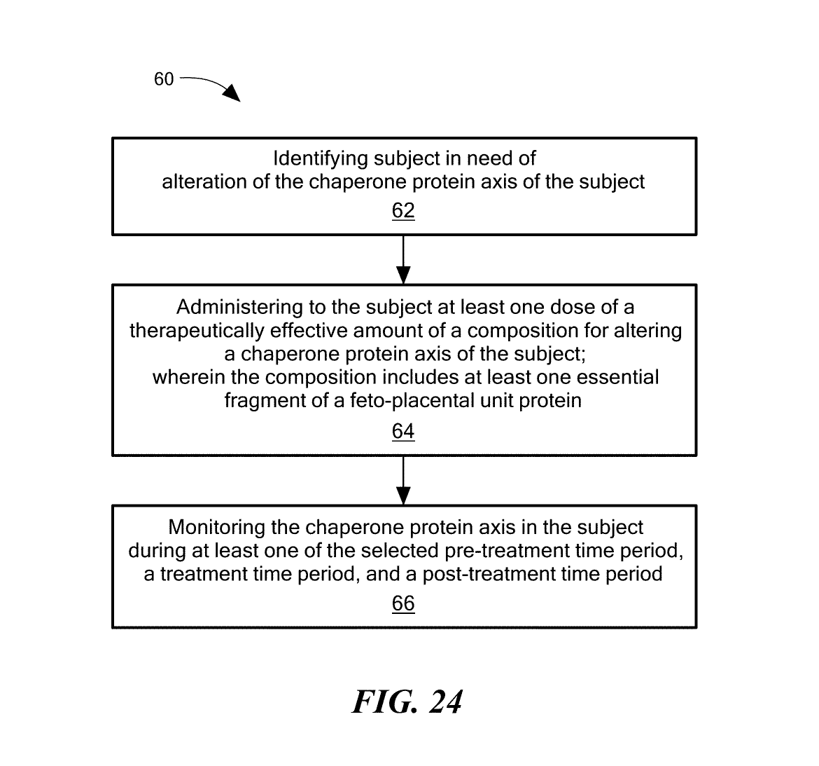

In one aspect, an inventive method 60 of treatment is shown in FIG. 24. The method 60 includes administering to the subject at least one dose of a therapeutically effective amount of a composition for altering a chaperone protein axis of the subject; wherein the composition includes at least one essential fragment of a feto-placental unit protein, as shown in step 64. In one embodiment, the method of treatment 60 of the invention can include identifying a candidate for treatment including identifying a subject in need of alteration of the chaperone axis of the subject, as shown in step 62. The identification can include, for non-limiting examples, pre-treatment evaluation of the past, present and possible future general or specific health status of a candidate subject including evaluation of the candidate subject's genetic make-up and predisposition for disease or other condition. The pre-treatment evaluation can also include a pre-treatment assessment of the chaperone protein axis of the subject. Such pre-treatment chaperone protein axis assessment can include measuring an intracellular or extracellular level of a key chaperone protein involved in the chaperone protein axis of the subject. In a preferred embodiment, the chaperone protein is a protein included in the HSP70 family. In a preferred embodiment, the feto-placental unit protein is a mammalian feto-placental unit protein.

In another embodiment, the treatment method 60 of the invention can include monitoring the chaperone protein axis of a subject including monitoring the chaperone axis in the subject during at least one of a selected pre-treatment time period, a treatment time period, and a post-treatment time period, as shown in step 66. Such monitoring can include measuring pre-treatment amount(s) of at least one of an intracellular and/or extracellular level of a key protein in the chaperone protein axis of the subject, and in addition, measuring at least one of such levels during and after the treatment for a select period of time. In a preferred embodiment, the key protein is a protein included in the HSP70 family.

The inventive treatment method can include altering the chaperone protein axis of the subject for the growth or regeneration of a cell in the subject. In addition, or alternatively, the method can include altering the chaperone axis of the subject for the repair of a cell in the subject.

The inventive treatment method includes at least one dose of a therapeutically effective amount of the composition. The therapeutically effective amount of the composition can include a range of 3 mg of the composition per 100 kg body weight of the subject to as much as 3 g of the composition per 100 kg body weight of the subject.

The method of treatment can include at least one of several administration methods, as discussed above. In addition, the method of treatment can include the administration of the composition with at least one additional component for optimizing receipt and utilization by the subject, and/or for achieving an additive and/or synergistic effect. The additional component can be added to and/or mixed with the composition prior to administration. Alternatively, the additional component can be administered simultaneously but separately from the composition. In yet another alternative, the additional component can be administered separately from the composition but consecutively with the composition either prior to or following administration of the composition and/or at a select time interval between administration of the composition and administration of the additional component.

Preferably the additional component includes an oil, and more preferably, the additional component includes an emu oil. More preferably, the composition includes a preparation of the essential fragment of the feto-placental unit being mixed with at least the additional component to form a paste. More preferably, the composition is administered transcutaneously to the subject.

The treatment method can include administering the at least one dose at a selected dosage rate over a selected dosing time interval. The selected dosage rate can include a range of one dose per one day to one dose per 365 days during a selected dosing time interval. The selected dosing time interval can include a range of a single day to a day substantially proximate to an expected end of life of the subject. The dosage rate and/or the dosing time interval can be based upon the one or more evaluation factors for the subject.

In the method of the invention, the chaperone protein axis level of a subject can be altered from a baseline or pre-alteration chaperone protein axis level to at least a minimum post-alteration chaperone protein axis level which is maintained for a sustaining period of time. The sustaining period of time can vary in a range of about 1 week to about 52 weeks. The minimum post-alteration chaperone protein axis level can be maintained for a sustaining period of time which continues after the treatment method has terminated and the composition is no longer being administered to the subject.

In different methods of the invention, the subject is a mammal, and preferably the mammal is a human and/or a horse.

The invention also features a pharmaceutical composition including a therapeutically effective amount of at least an essential fragment of a feto-placental unit protein for altering a chaperone protein axis in a subject. In addition, the invention can include a kit including the pharmaceutical composition. In a preferred embodiment, the feto-placental unit protein is a mammalian feto-placental unit protein.

In a subject which is human, the therapeutically effective amount is in a range from about 10 ug feto-placental unit protein/kg body weight to 1 mg feto-placental unit protein/kg body weight in the subject. In a subject which is a horse, the therapeutically effective amount is in a range from about 1 ug feto-placental unit protein/kg body weight to 1 mg feto-placental unit protein/kg body weight in the subject.

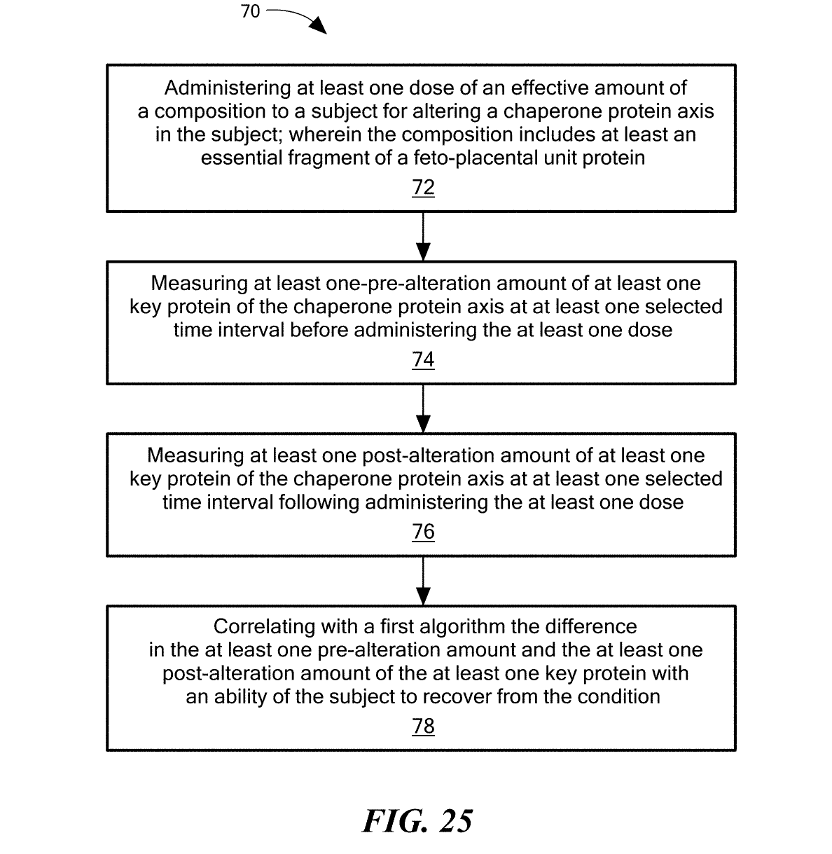

In another aspect, the invention also features a method 70 of assessing an ability of a subject to recover from a condition, as shown in FIG. 25. The condition may be caused by a defect or a disease, injury or other trauma. The assessment method 70 includes administering at least one dose of an effective amount of a composition to a subject for altering a chaperone protein axis in the subject, as shown in step 72. The composition includes at least an essential fragment of a feto-placental unit protein, also as shown in step 72. In a preferred embodiment, the feto-placental unit protein is a mammalian feto-placental unit protein. The method 70 further includes measuring pre-alteration and post-alteration amounts of at least one key protein of the chaperone protein axis at selected time intervals respectively before and after administration of the composition to the subject, as shown respectively in steps 74 and 76. The method 70 further includes correlating with a first algorithm the difference in the pre-alteration and post-alteration amounts of the key protein with an ability of the subject to recover from the condition, as shown in step 78.

The condition can be selected from the group consisting of a tendon injury, a muscle injury and at least one aging condition.

The essential fragment of the feto-placental unit protein includes an essential fragment of a chaperone protein and preferably, the chaperone protein includes a HSP70 family protein.

The effective amount of the composition is in a range of 1 ug composition to 10 g composition per 50 kg body weight of the subject.

The assessment method can include at least one of several administration procedures, as discussed above. The assessment method can include the administration of the composition with at least one additional component for optimizing receipt and utilization by the subject, and/or for achieving an additive and/or synergistic effect. The additional component can be added to and/or mixed with the composition prior to administration. Alternatively, the additional component can be administered simultaneously but separately from the composition. In yet another alternative, the additional component can be administered separately from the composition but consecutively with the composition either prior to or following administration of the composition and/or at a select time interval between administration of the composition and administration of the additional component.

Preferably, the composition of the assessment method is administered using a transcutaneous administration of the composition in combination with at least one additional component for achieving an additive or synergistic effect. Preferably, the additional component is an oil, and more preferably the additional component is an emu oil. More preferably, the composition includes the essential fragment of the feto-placental unit being mixed with the additional component in a paste.

The assessment method includes administering the composition at a selected dosage rate over a selected dosing time interval. The selected dosage rate can include a range of one dose per one day to one dose per 365 days during a selected dosing time interval. The selected dosing time interval can include a range of a single day to a day substantially proximate to an expected end of life of the subject. The dosage rate and/or the dosing time interval can be based upon the one or more evaluation factors for the subject.

The assessment method can include a mammalian subject and preferably a subject which is a human and/or a horse.

The invention also features a kit for assessing an ability of a subject to recover from a condition. The condition can be related to a disease, injury or other trauma. The kit includes a composition for altering a chaperone protein axis of the subject. The composition includes at least an essential fragment of a feto-placental unit protein. The kit also includes a device for administering the composition. In a preferred embodiment, the feto-placental unit protein is a mammalian feto-placental unit protein.

The kit can also include one or more devices for measuring at least one pre-alteration amount and at least one post-alteration amount of at least one key protein of the chaperone protein axis at at least one selected time interval respectively before and after the composition is administered to the subject.

The kit can also include at least one device for correlating with a first algorithm the difference in the pre-alteration and post-alteration amount(s) of the key protein(s) with an ability of the subject to recover from the condition.

The feto-placental unit proteins including at least one essential fragment of a feto-placental protein are harvested, processed, stored, and administered according to the method and composition disclosed in patent application numbers U.S. Ser. No. 14/330,818 and U.S. Ser. No. 14/551,612. Such method and composition are described below.

Method for Harvesting, Processing, and Storage of Proteins from the Mammalian Feto-Placental Unit and Use of Such Proteins in Compositions and Medical Treatment

### Background:

The method and composition broadly relates to methods of harvesting mammalian feto-placental proteins and in particular, chaperone proteins, for use in compositions and medical therapies for the treatment of disease or aging in mammals, and in particular, to humans.

Aging, which is both chronological and determined by cellular processes, is associated with an increase in disease and pathological processes. The causes of aging are unknown, but recent reviews correlate aging in humans with the instability of mitochondria leading to an increase in intra-cellular reactive oxygen species (ROS), potential shortening of the telomere, protein dysfunction and/or cellular death. The increase in ROS in turn appears to adversely affect DNA, mRNA, and protein synthesis, folding, transport and degradation. The culmination of these events in turn leads to apoptosis, i.e., cellular “suicide”, followed by necrosis, i.e., cellular death.

Some research has focused on mechanisms which initiate or allow the up-regulation of intra-cellular ROS to determine whether the mechanisms are sudden or cumulative and whether the mechanism can be slowed to any extent. Research has also investigated the role of chaperone proteins in responding to increased ROS. Notably, aging appears to decrease the intra-cellular and extra-cellular levels of chaperone proteins. For the purposes of this application, chaperone proteins, which include pharmacological chaperones, pharmacoperones, and pharma-cochaperones, are defined as target-specific, small molecules that bind to their target proteins to facilitate biogenesis and/or prevent and/or correct protein misfolding. Pharmacological chaperoning: a primer on mechanism and pharmacology, Leidenheimer Nancy J., Ryder Katelyn G., Pharmacol Res. 2014 May; 83:10-9. doi: 10.1016/j.phrs.2014.01.005. Epub 2014 Feb. 14. Chaperone protein targets include enzymes, receptors, transporters, and ion channels. Id. Chaperone proteins prevent the accumulation of misfolded proteins by promoting their refolding, degeneration and/or exocytosis. Keep your heart in shape: molecular chaperone networks for treating heart disease, Tarone Guido, Brancaccio Mara, Cardiovasc Res. 2014 Jun. 1; 102(3):346-61. doi: 10.1093/cvdcvu049. Epub 2014 Feb. 28., PMID:24585203 [PubMed—in process]. Chaperone proteins also play a role in intracellular signaling by controlling conformational changes required for the activation and deactivation of signaling proteins and assembly in signalosome complexes. Id.

Today, chaperone proteins are not readily available for medical research and possible medical therapies, however. Thus, there is a need for methods of harvesting, processing and storing chaperone proteins. In addition, there is a need to develop compositions and medical therapies which make use of chaperone proteins in the treatment of disease or aging, and in particular, disease related to protein dysfunction.

### Brief Summary:

In one aspect, a method of harvesting, processing and storing a plurality of proteins from a mammalian feto-placental unit is provided. The method includes dissecting a mammalian uterus to harvest at least one component of the mammalian feto-placental unit; blast freezing the component; and storing the blast frozen component; wherein the blast frozen component includes the plurality of mammalian feto-placental unit proteins. In one embodiment, the method includes lyophilizing the blast frozen component to remove at least some water from the blast frozen component thereby creating a freeze-dried form; and storing the lyophilized component; wherein the lyophilized component includes the mammalian feto-placental unit proteins. In an additional embodiment, the mammalian feto-placental unit proteins include at least one essential fragment of at least one of the mammalian feto-placental unit proteins. In a further additional embodiment, the mammalian feto-placental unit proteins include a plurality of chaperone proteins.

In another embodiment, the plurality of mammalian feto-placental unit proteins are selected from the group consisting of serum albumin, actin, cytoplasmic 1, I alpha globin, hemoglobin fetal subunit beta, vimentin, beta globin chain, TPM1, annexin A2, protein disulfide isomerase family A member 3, alpha-2-HS-glycoprotein, fatty acid binding protein 5, cofilin-1, 78 kDa glucose-regulated protein, gelsolin isoform b, beta-A globin chain, glyceraldehyde-3-phosphate dehydrogenase, heat shock protein alpha, heat shock protein 70, peptidylprolyl isomerase A, 14-3-3 protein zeta/delta, histone H3, peroxiredoxin 2, cathepsin D, uterine milk protein, tubulin beta chain, myosin light chain 6, endoplasmic reticulum protein 29, tubulin alpha chain, solute carrier family 2, facilitated glucose transporter member 1, alpha-1-antitrypsin transcript variant 1, heat shock protein 10, pregnancy-associated glycoprotein 3, hemoglobin subunit beta, isocitrate dehydrogenase [NADP] cytoplasmic, elongation factor-1 alpha, phosphoglycerate kinase, 14-3-3 protein epsilon, putative tropomyosin, tumor protein translationally-controlled 1, galectin-1, transaldolase 1, pregnancy-associated glycoprotein 4, pregnancy-associated glycoprotein 1, sodium/potassium-transporting ATPase subunit alpha-1, lamin B1, pregnancy-associated glycoprotein 6, 14-3-3 protein beta/alpha, metallopeptidase inhibitor 2, fatty acid binding protein 5, myosin regulatory light chain MRCL3, transferrin, enolase 1, cathelicidin-1, 6-phosphogluconate dehydrogenase decarboxylating, elongation factor-1 alpha, ATP-citrate synthase, ribosomal protein S8, pyruvate kinase, pre-mRNA splicing factor SRP20-like protein, alpha 2, 5 prime, malate dehydrogenase, cystatin-B, chorionic somatomammotropin hormone, carbonic anhydrase 2, SLC25A6, decorin, 60S ribosomal protein L6, protein disulfide isomerase-associated 4, pregnancy-associated glycoprotein 11, prostaglandin F synthase, integrin beta-1, H+ transporting ATP synthase subunit D, RHOA, adenylate kinase, lactate dehydrogenase A, RAB10, glucose-6-phosphate 1-dehydrogenase, elongation factor 1-delta, ribosomal protein S17, insulin-like growth factor-binding protein-7, ribosomal protein L19, ATP synthase alpha subunit, RAC1, calpain II 80 kDa subunit, secreted phosphoprotein 24, CD9 antigen, aspartate aminotransferase, DNA mismatch repair protein MutL, ribosomal protein S6, 14-3-3 protein gamma LDHA protein, putative peptidase, myosin light chain kinase, smooth muscle, cAMP-dependent protein kinase regulatory subunit alpha 1, elongation factor 1-alpha, actin, GNAZ, eukaryotic translation initiation factor 5A, mitochondrial bifunctional methylenetetrahydrofolate dehydrogenase/cyclohydrolase, solute carrier family 2, facilitated glucose transporter member 3, thioredoxin, ADP-ribosylation factor 1, NADH dehydrogenase (ubiquinone) 1 beta, proteasome subunit alpha type, 2, gamma fibrinogen, putative H-ATPase subunit B, proteasome subunit alpha type, 60S ribosomal protein L10, 14-3-3 protein sigma, chaperone protein DnaK, ribosomal protein s15, putative uncharacterized protein, aspartyl-tRNA synthetase, and proteasome subunit alpha.

In another embodiment, the method includes using at least a portion of the mammalian feto-placental unit proteins for medicinal purposes. In an additional embodiment, the step of using at least a portion of the mammalian feto-placental unit proteins for medicinal purposes includes treating for cellular repair in a mammalian subject. In another embodiment, the step of using at least a portion of the mammalian feto-placental unit proteins for medicinal purposes includes treating for a disease or aging in a mammalian subject. In still other embodiments, the treating step includes administering the portion of the mammalian feto-placental unit proteins to the mammalian subject with at least one of a sublingual procedure, an intra-ocular procedure, an intra-rectal procedure, and an intra-gastro-intestinal procedure.

In an additional embodiment, the treating step includes reconstituting the portion of the mammalian feto-placental unit proteins of the lyophilized component with a fluid; and administering the reconstituted portion of the mammalian feto-placental unit proteins to the mammalian subject. In another embodiment, the fluid is an oil. In further additional embodiments, the administering step includes administering the reconstituted portion of the mammalian feto-placental unit proteins to the mammalian subject through a procedure selected from the group consisting of an oral administration, a rectal administration, a cutaneous administration, a subcutaneous administration, an intravenous injection, and an intramuscular injection. In a preferred embodiment, the administering procedure is a cutaneous administration

In another embodiment, the component is selected from the group consisting of a placenta-cord fetal component, a liver component, a spleen component, a whole brain component, an ocular component, a gastro-intestinal component, a female specific component, and a male specific component.

In another aspect, a composition is provided. The composition includes a plurality of mammalian feto-placental unit proteins from at least one lyophilized, blast frozen component of a harvested mammalian feto-placental unit. In one embodiment, the mammalian feto-placental unit proteins include at least one essential fragment of at least one of the mammalian feto-placental unit proteins. In another embodiment, the mammalian feto-placental unit proteins include a plurality of chaperone proteins. In yet another embodiment, the lyophilized, blast frozen component is selected from the group consisting of a placenta-cord fetal component, a liver component, a spleen component, a whole brain component, an ocular component, a gastro-intestinal component, a female specific component, and a male specific component.

In an additional aspect, a method of treatment of a disease or aging in a mammalian subject is provided. The method includes administering to the mammalian subject a plurality of mammalian feto-placental unit proteins from at least one blast frozen component harvested from a mammalian feto-placental unit; and reducing an accumulation of at least one intracellular protein in the mammalian subject. In one embodiment, the mammalian feto-placental unit proteins include at least one essential fragment of at least one of the mammalian feto-placental unit proteins. In another embodiment, the mammalian feto-placental unit proteins include a plurality of chaperone proteins. In yet another embodiment, the step of reducing the accumulation of the intracellular protein comprises at least one of folding at least a portion of the intracellular protein, refolding at least a portion of the intracellular protein, degrading at least a portion of the intracellular protein and transferring at least a portion of the intracellular protein across a cellular membrane.

### Detailed Description:

Methods for harvesting proteins or essential fragments thereof, and in particular chaperone proteins, from the mammalian feto-placental unit, and the use of such mammalian feto-placental unit proteins in compositions and medical therapies for the treatment of disease and aging are discussed.

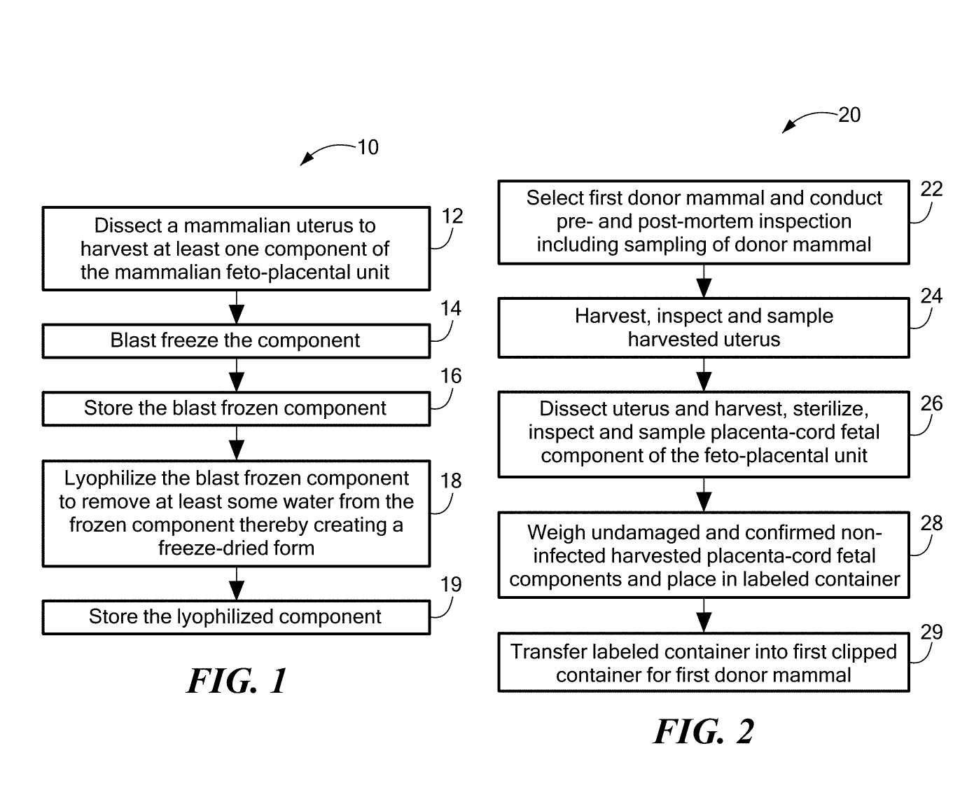

In one aspect, a method 10 of harvesting, processing and storing proteins from a mammalian feto-placental unit, is shown in FIG. 1. The method includes the steps of dissecting a mammalian uterus to harvest at least one component of the mammalian feto-placental unit, as shown in step 12; and blast freezing the component, as shown in step 14; wherein the component includes the proteins. In one embodiment, the method includes storing the blast frozen component, as shown in step 16. In another embodiment, the method includes lyophilizing the blast frozen component to remove at least some water from the frozen component thereby creating a freeze-dried form, as shown in step 18; and storing the lyophilized component, as shown in step 19.

Further embodiments of the method 20 are shown in FIG. 2. A female pregnant mammal, such as, for example, a pregnant ewe, is selected as a first donor mammal for supplying a uterus, as shown in step 22. A registered veterinarian conducts a pre-mortem and post-mortem examination of the selected first donor mammal to ensure that the donor is healthy and free of any visible disease. For example, the veterinarian inspects for muscular wasting which in an ovine could indicate parasitism or Johnes disease. The registered veterinarian also inspects for normal motor-function to rule out any central nervous system disease, such as, for example, aberrant larval migration, spinal cord and/or brain infection including infection in the middle and inner ear. The registered veterinarian also conducts a post-mortem examination of the selected first donor mammal to confirm the health and absence of disease in the donor mammal. The veterinarian can inspect for, for example, enlarged kidneys and nephrosis which could indicate a clostridial type infection. Core samples are taken from the liver and/or spleen of the first donor mammal for bacterial and viral isolation. For purposes of the method, only tissue negative to bacterial and viral isolation are harvested.

The uterus is harvested and inspected by the registered veterinarian to confirm the absence of lesions and/or other defects in the uterus, as shown in step 24. The uterus is dissected and the placenta with cotyledons and umbilical cord are separated from the remaining fetal mass and washed in a sterile non-pyrogenic solution containing no antimicrobial agents such as, for example, Lactated Ringers Solution (LRS), as shown in step 26. The harvested placenta-cord fetal components are inspected for damage such as, for example, tissue bruising or tearing which may have occurred during harvesting and dissection. Samples of the undamaged harvested placenta-cord fetal components are analyzed for infection using bacterial and/or viral cultures. Undamaged, confirmed non-infected harvested placenta-cord fetal components are weighed and placed in a labeled sterile sample container, as shown in step 28. The sterile sample container holding the placenta-cord fetal components is then transferred into a first clipped, that is, securely closed, sterile container, as shown in step 29 of FIG. 2. Damaged and/or infected dissected placenta-cord components are discarded.

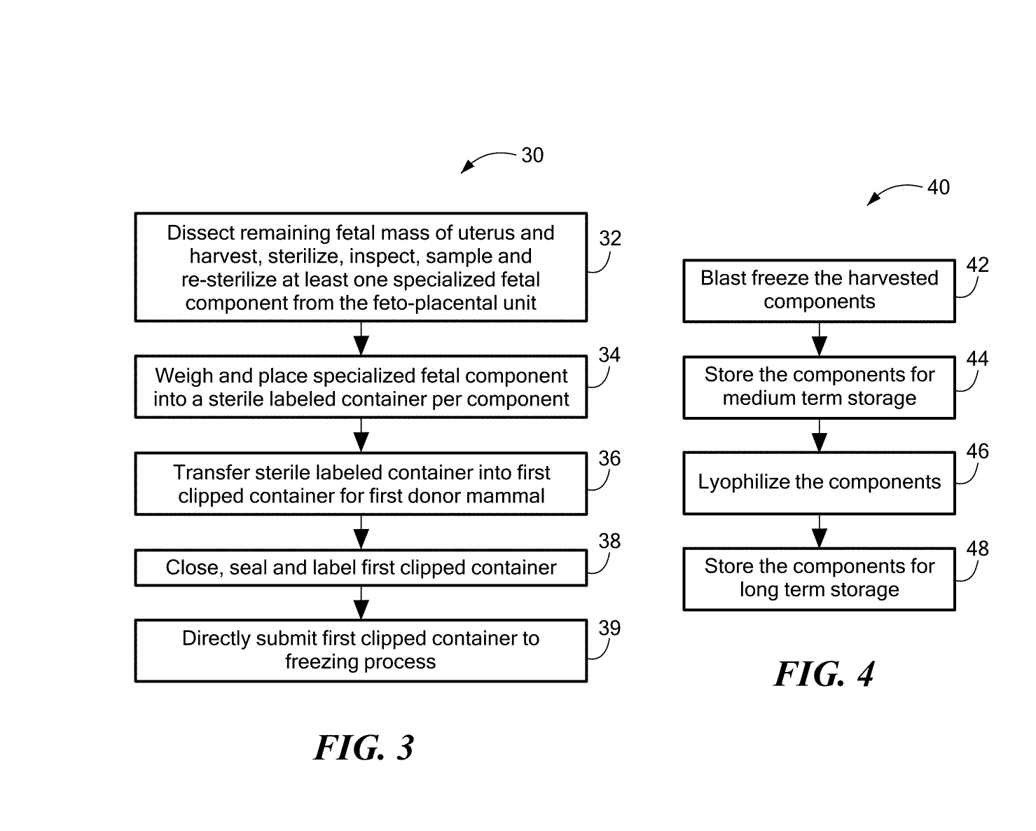

Further embodiments including the steps of method 30 are illustrated in FIG. 3. The remaining fetal mass not including the placenta-cord fetal components is also washed in a sterile non-pyrogenic solution containing no antimicrobial agents such as, for example, LRS, and transferred to a dissection table, as shown in step 32. Under sterile conditions, the sterilized remaining fetal mass is dissected into relevant specialized fetal components such as, for example, a liver component, a spleen component, a whole brain component, an ocular component, a gastro-intestinal component, a female specific component which includes accessory sexual glands, and a male specific component which includes male penile tissue, testicular tissue and accessory sexual glands. The harvested specialized fetal components are inspected for damage during dissection. Samples of undamaged harvested specialized fetal components are analyzed for infection using bacterial and/or viral cultures. Damaged and/or infected dissected specialized fetal components are discarded. Non-damaged, non-infected harvested specialized fetal components are again washed in a similar sterile, non-pyrogenic solution, such as, for example, LRS. Each re-sterilized harvested specialized fetal component is weighed and placed in its own sterile labeled container per component, as shown in step 34. The labeled containers holding the harvested specialized fetal components corresponding to the same first donor mammal are then transferred into the first clipped sterile container along with the placenta-cord fetal components for the same first donor mammal, as shown in step 36.

The first sterile clipped container is closed, sealed and labeled with identification information including weight and any notes regarding the dissection as shown in step 38. The prior multiple inspections of the entire feto-placental unit ensure that normal fetal growth has occurred and that there is no sign of infection and concomitant disease. The first sterile clipped container is directly submitted to a process for freezing the harvested components, as shown in step 39.

The freezing and storage processes 40 of the method are shown in FIG. 4. The freezing process is accomplished using a fast or rapid freezing otherwise known as a blast freezing, as shown in step 42. The blast freezing lowers the temperature of the components to at least −6° C., and preferably to at least −10° C., and more preferably to at least −20° C., in eight hours, and preferably in six hours, and more preferably in four hours and most preferably in two hours.

After the blast freezing temperature is reached, the components can be stored in a freezer in medium term storage, as shown in step 44, for up to twelve months, and preferably for up to six months, and more preferably for up to three months, and most preferably for up to two months. In medium term storage, the temperature of the components is maintained between −20° C. and −100° C., and preferably between −40° C. and −90° C., and more preferably −60° C. and −80° C., and most preferably between −65° C. and −75° C.

Immediately following blast freezing, or following blast freezing and medium term storage as shown in step 44 of FIG. 4, the components can be further lyophilized or freeze dried to remove water from the components, as shown in step 46, using methods known to those of ordinary skill in the art. A freeze dried form of the component is thereby formed. Lyophilization is conducted on a batch basis by weight and according to type of component. Thus, the placenta-cord fetal component and the specialized liver, spleen, brain, ocular, gastro-intestinal, and female and male specific fetal components are lyophilized by weight according to component type to satisfy minimum weight requirements. Components are lyophilized to weigh a minimum of 10 grams of specific tissue, and preferably a minimum of 8 grams of specific tissue, and more preferably a minimum of 6 grams of specific tissue, and most preferably a minimum of 4 grams of specific tissue.

The components can then be stored for long term storage in their lyophilized, that is, freeze-dried form, as shown in step 48. The temperature of long term storage is maintained between 5° C. and 30° C., and preferably between 10° C. and 25° C., and more preferably between 15° C. and 20° C., and most preferably at 20° C. The duration of long term storage can equal as much as 100 years, and preferably a maximum of 50 years, and more preferably a maximum of 10 years, and even more preferably a maximum of 2 years, and most preferably a maximum of 1 year.

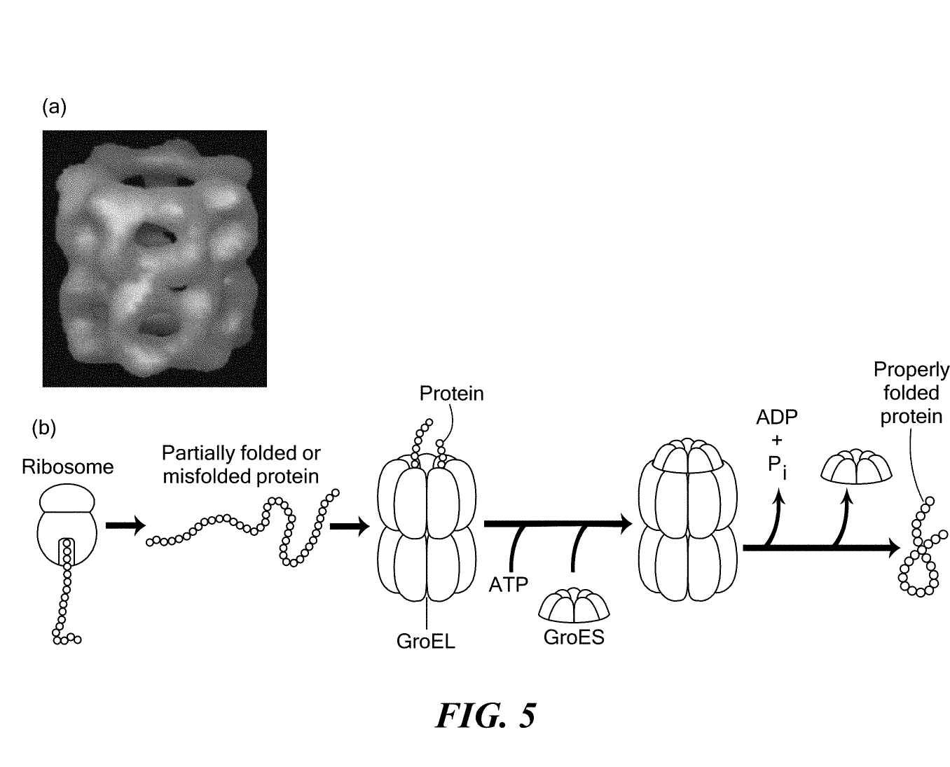

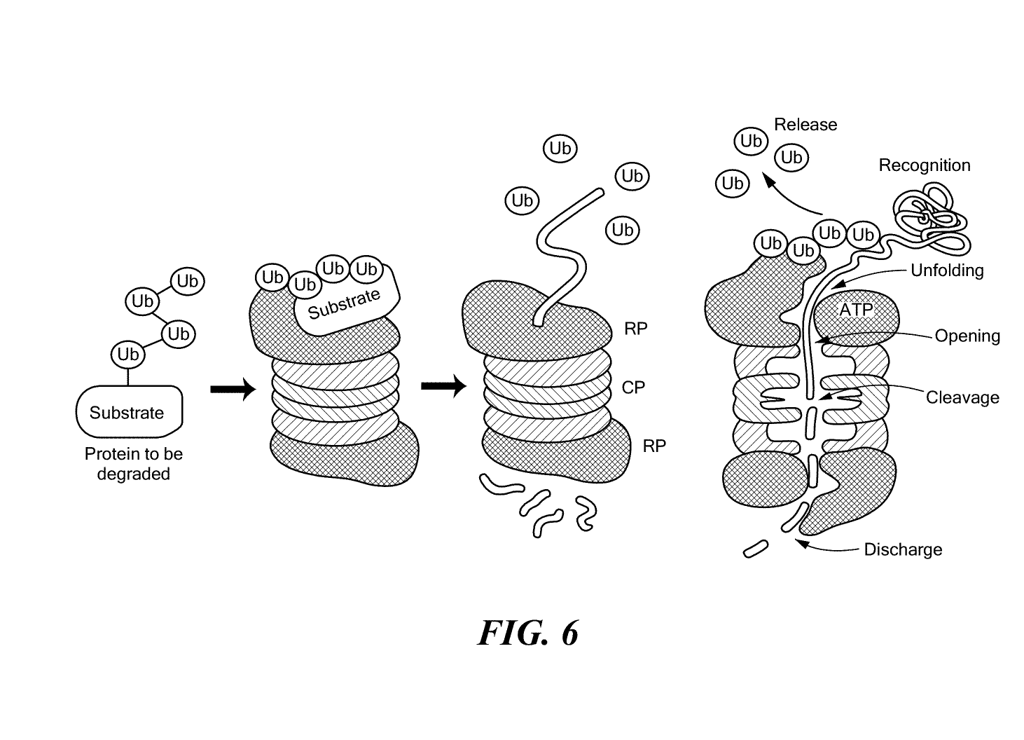

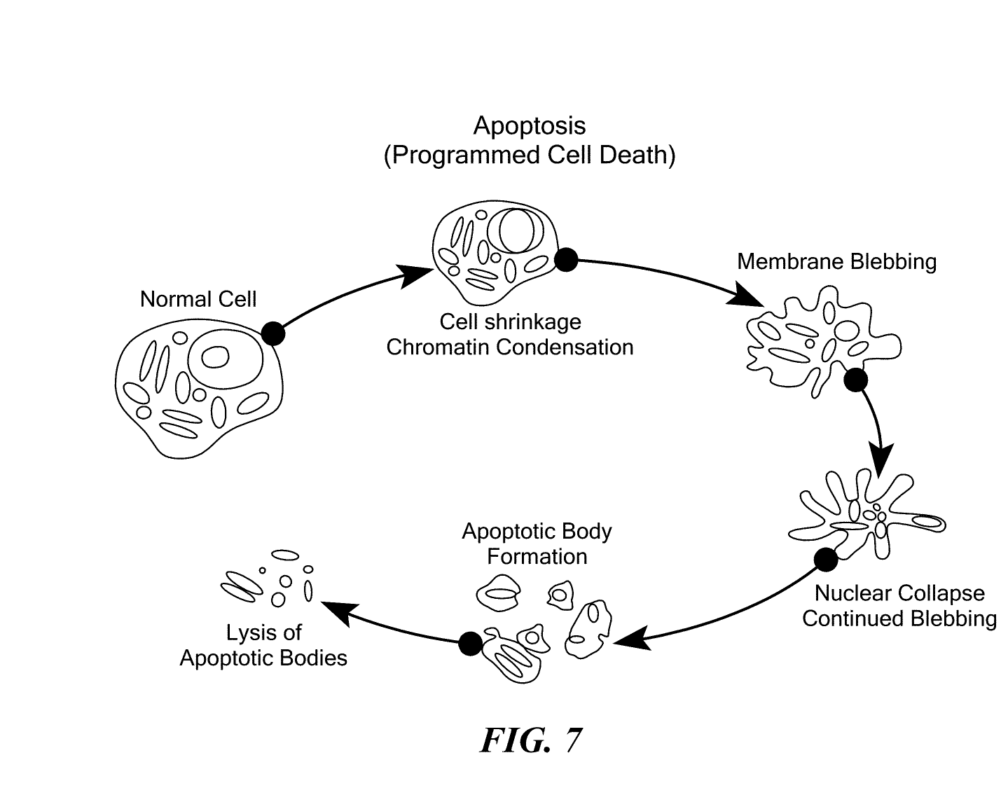

The harvested and processed mammalian fetal placental unit components include a plurality of proteins or essential fragments thereof. In one embodiment, the mammalian feto-placental unit protein or essential fragments include chaperone proteins. The chaperone proteins can have the ability to re-fold wrongly folded proteins as shown in FIG. 5. In addition or alternatively, the chaperone proteins can degrade otherwise not fully metabolized proteins. FIG. 6 demonstrates this mechanism, where UB identifies ubiquitors, CP identifies chaperone proteins, and RP identifies residual proteins. Furthermore, in addition or alternatively, the chaperone proteins can reverse cellular apoptosis, as shown in FIG. 7. As a cell descends into the spiral of cell death, the cell most commonly goes through a biologic sequence of cellular shut down mechanisms. The shutdown mechanisms take various forms but in essence lead to intracellular protein accumulation mainly through a lack of protein transport out of the cell. The protein accumulation in turn leads to osmotic imbalance, cell membrane disruption and cell membrane disintegration. Chaperone proteins can work intracellularly to fold, refold, transport or degrade accumulated proteins. The reverse of toxic protein accumulation ameliorates osmotic imbalance thereby preventing cellular membrane disruption. As a result, the cellular death spiral is reversed. Further, in addition or alternatively, the chaperone proteins can affect intracellular signaling by controlling conformational changes required for activation or deactivation of signaling proteins, and their assembly in specific signalosome complexes. See Keep your heart in shape: molecular chaperone networks for treating heart disease, Tarone Guido, Brancaccio Mara, Cardiovasc Res. 2014 Jun. 1; 102(3):346-61. doi: 10.1093/cvr/cvu049. Epub 2014 Feb. 28., PMID:24585203 [PubMed—in process] at abstract.

Accordingly, in embodiments of the method, the proteins of the components of the mammalian feto-placental unit can be used for medicinal purposes in the treatment of disease and/or aging in mammalian subjects including, for example, humans. Most disease and aging is characterized by some form of cellular dysfunction including the slowing of cellular mechanisms and the buildup of dysfunctional proteins within the cell. This cellular dysfunction leads to cellular under performance and clinical manifestation of disease and/or aging. The proteins of the components of the mammalian feto-placental unit including essential fragments and chaperone proteins can be administered to promote folding, refolding, transport or degradation of accumulated proteins within cells.

An example of a disease mechanism which can be targeted by the method is the manifestation of type 2 diabetes. Although this disease can have multi-centric etiologies and manifestations, the cellular basis of the disease relates to the compromised ability of the cell to make insulin receptors and/or to respond to insulin/insulin receptor interaction because of the accumulation of proteins. The compromised cellular abilities lead to a lack of systemic glucose regulation which in turn leads to a cascade of multi-compartmental manifestations of clinical symptoms, but most specifically to an interference with arteriole circulation. The arteriole circulation interference leads to a decrease in blood circulation, vascular constriction, rise in blood pressure, and a decrease in vascular support for organs and cells. Ischemia, cellular apoptosis and eventually cell death result.

The method including the administration of mammalian feto-placental unit proteins to a mammalian subject such as a human can counteract and at least partially reverse this cascade of events. The chaperone proteins administered to the human subject are from an exogenous biologic source of mammalian feto-placental tissue. After administration to the subject, the mammalian feto-placental unit proteins including chaperone proteins can work intracellularly to fold, refold, transport, and/or degrade accumulated proteins. These mechanisms at least partially reverse the toxic accumulation proteins. As a result, the cellular death spiral is reversed at least partially leading to the return of cellular function related to the response of the insulin/insulin receptor complex, the relaxation of arterioles, the decrease in blood pressure, the re oxygenation of tissue, the reverse of ischemia and the return to normal cell function and organ function.

At least portions of the tissues including the proteins can be extracted from the blast frozen components of the mammalian feto-placental unit, thawed and directly administered to the mammalian subject using different procedures employing, for example, a spatula, a sponge, a gel, and encapsulation. Non-limiting procedures for protein administration to a mammalian subject include a sublingual procedure, an intra-ocular procedure, an intra-rectal procedure and an intra-gastro-intestinal procedure.

Alternatively, at least portions of tissues including the proteins can be extracted from the lyophilized components of the mammalian feto-placental unit and reconstituted with a fluid, such as, for example, sterilized water or saline. The reconstituted proteins can be administered to the mammalian subject using various procedures such as, for non-limiting examples, an oral administration, a rectal administration, a cutaneous administration, a subcutaneous administration, an intravenous injection, and an intramuscular injection.

The proteins including the essential fragments thereof harvested from the mammalian feto-placental unit, processed and stored according to the method are included in the list provided in Table 1. All versions of the database: the uniprot-_20130128_5wcYYr database are included for the purposes of the specification.

Examples of various diseases and/or symptoms characteristic of aging which can be treated by the proteins including the essential fragments thereof harvested from the mammalian feto-placental unit, and processed according to the method include, for example, type 2 diabetes, type 1 diabetes, cardiomyopathy, prostate cancer, renal dysfunction, and intra- and extra-cellular storage disease, Alzeimer disease, Parkinson disease, sexual dysfunction including female dysfunction (e.g., fluid reduction) and male dysfunction (e.g., erectile dysfunction), hormonal imbalance including hormonal imbalance around the cessation of mensus or menopause, musculoskeletal disorder, and neoplasia. Selected proteins are known to those of ordinary skill in the art to ameliorate selected specific diseases as discussed in, for example, Growth charts for patients with Hunter syndrome, Patel P, Suzuki Y, Maeda M, Yasuda E, Shimada T, Orii K E, Orii T, Tomatsu S., Mol Genet Metab Rep. 2014; 1:5-18, PMID: 24955330, [PubMed], and other articles identified in http://www.ncbi.nlm.nih.gov/pubmed/?term=mucopolysaccharide+storage+disease. Accordingly, with respect to the treatment of extra-cellular storage diseases, in another embodiment of the method, mammalian feto-placental unit proteins are administered to a mammalian subject for the treatment of extracellular storage diseases involving the toxic accumulation of extra-cellular proteins. The mammalian feto-placental proteins act to reduce the toxic accumulation of extra-cellular proteins by degrading, folding, refolding and/or transferring the toxic proteins across cellular membranes. In still other embodiments of the method, mammalian feto-placental unit proteins are administered to a mammalian subject for the treatment of intra- and extra-cellular storage diseases involving the toxic accumulation of mucopolysaccharides. Mucopolysaccharides, also known as glycosaminoglycans, consist of long chains of sugar molecules. In a normally functioning individual, enzymes are produced to break down the mucopolysaccharides into simpler molecules which the body can then utilize. In individuals suffering from mucopolysaccharide storage diseases, either the individuals do not produce sufficient quantities of the necessary enzymes to break down the mucopolysaccharides into smaller molecules for use by the body, or the individual produces enzymes which are defective and unable to break down the mucopolysaccharides into the necessary smaller molecules. Thus, mucopolysaccharides can accumulate in cells, connective tissue and/or the blood stream and lead to severe disease and death. Such individuals are traditionally treated with exogenous enzymatic therapies for breaking down the mucopolysaccharides. In the methods, mammalian feto-placental proteins can be administered to the mammalian subject, and the mammalian feto-placental proteins can break down the mucopolysaccharides into smaller molecules thereby reducing the toxic accumulation of such long chain sugars.

In another aspect, a composition including proteins originating from at least one blast frozen, lyophilized component of a harvested mammalian feto-placental unit is provided. In one embodiment, the proteins include at least one essential fragment of at least one of the proteins. In another embodiment, the proteins include chaperone proteins. In yet other embodiments, the component is selected from the group consisting of at least one of a placenta-cord fetal component, a liver component, a spleen component, a whole brain component, an ocular component, a gastro-intestinal component, a female specific component, and a male specific component.

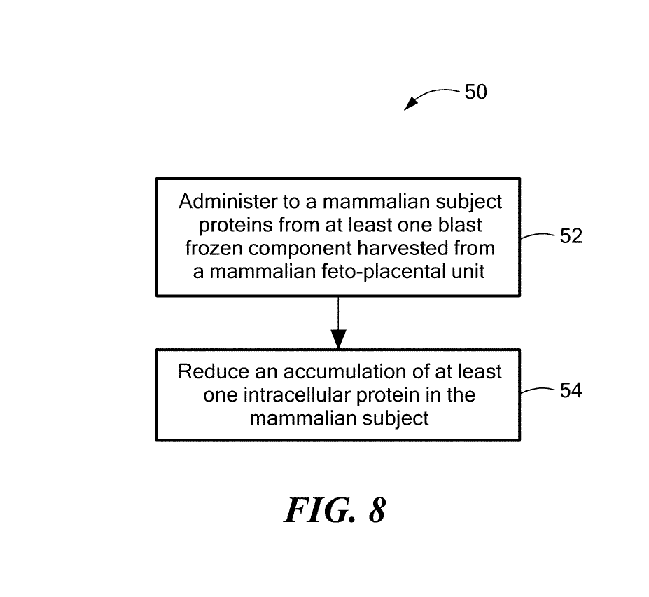

An additional aspect is shown in method 50 in FIG. 8 which provides for the treatment of a disease or aging in a mammalian subject. The method includes administering to the mammalian subject a plurality of proteins from at least one blast frozen component harvested from a mammalian feto-placental unit, as shown in step 52; and reducing an accumulation of at least one intracellular protein in the mammalian subject, as shown in step 54. In one embodiment, the step of reducing the accumulation of the intracellular protein includes at least one of folding, refolding, transfer, and degradation of the intracellular protein.

### EXAMPLE 1

### Prior Art Method for Harvesting, Processing, and Storage of Proteins from the Mammalian Feto-Placental Unit and Use of such Proteins in Compositions and Medical Treatment

A donor sheep certified according to New Zealand Protocol was selected. Placenta-cord, liver, gastro-intestinal and specific male fetal components were dissected, harvested, and processed including inspection, sterilization, blast freezing followed by lyophilization. The lyophilized components were stored for at least of two years. Tissue samples were then extracted from the lyophilized components periodically according to the patient treatment protocol and reconstituted with sterilized saline to form 6 ml injection samples including 1 gram of reconstituted lyophilized component per each sample.

At the onset of treatment, Patient X was in his early nineties and had concomitant prostatic cancer which exhibited as an enlargement of the prostate gland coupled with a prostate specific antigen (PSA) score of over 20. The patient had experienced baldness for approximately 40 years. The patient suffered from clinical stiffness and exhibited stooped posture due to osteoporosis. The patient reported experiencing erectile dysfunction and low libido.

The injection samples including the lyophilized components reconstituted with sterilized saline were injected subcutaneously into Patient X once per month for 6 months and once per 3 months thereafter for 5 years.

After two months of treatment, the patient's clinical physician observed a PSA of 15 and a decrease in prostatic gland size to 30% of the pre-treatment size. Subsequently, the patient's PSA dropped to less than 5 and remained less than 5 thereafter. New hair growth was observed on top of the patient's head. The patient no longer suffered from pre-treatment clinical stiffness and maintained an erect posture. The patient reported return of libido and erectile function.

### EXAMPLE 2

The effect of the administration of a composition of the invention on the level of extracellular HSP70 chaperone protein circulating in the blood stream was studied in eight male and two female human subjects (8 male, 2 female) aged 40 to 80 years of age. At the onset of the study, the subjects did not exhibit signs of ongoing clinical disease other than some frailty and inflammation substantially typical or characteristic of each individual's age. Immediately, prior to any treatment, that is at Day 0, the level of extracellular HSP70 proteins circulating in the blood stream of each of the subjects was measured by using an Elisa kit for protein detection and quantification.

The composition of the invention was prepared by adding an emu oil to a portion of a dry portion of mammalian feto-placental unit which had been harvested and dissected from a ewe including its fetus. The composition was prepared including a ratio of 1 part powder to 9 parts emu oil. The powder and oil were mixed to form a homogenous paste. Each administration dose was prepared including 300 mg power and 3 ml of emu oil.

The composition of the invention was administered using a transcutaneous procedure to the forearm of each subject at a dosage rate of one treatment per week for a total of four consecutive weeks. At Day 35, the level of extracellular HSP70 proteins circulating in the blood stream of each of the subjects was then measured by using an Elisa kit for protein detection and quantification.

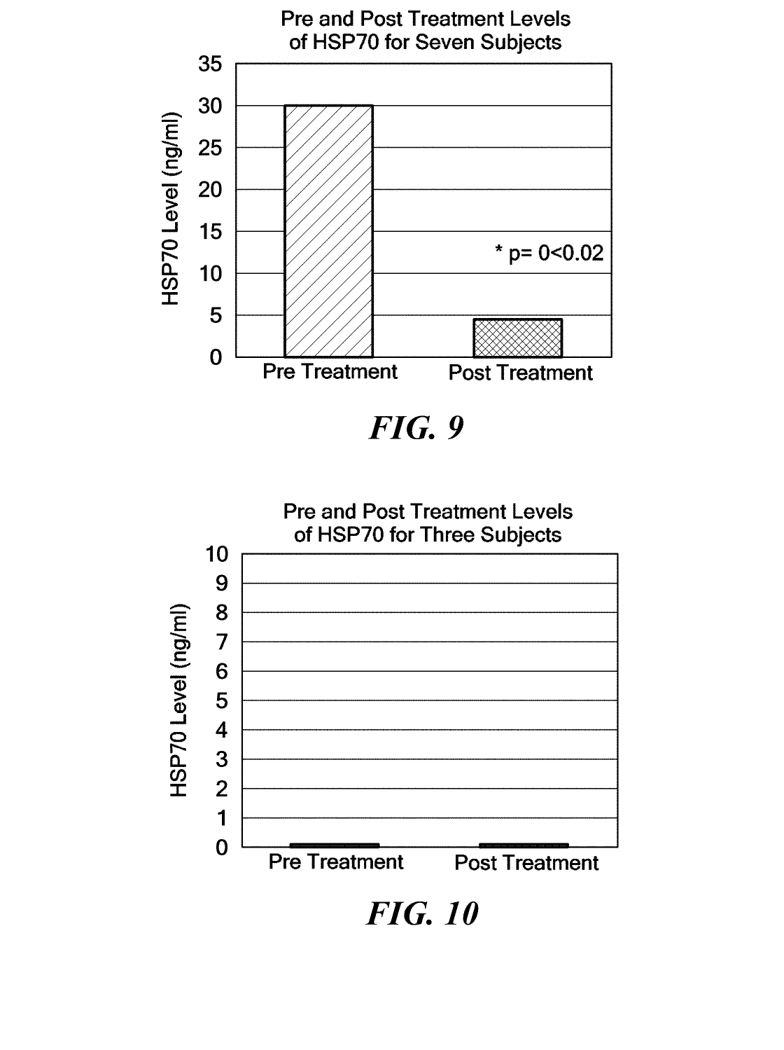

FIGS. 9-11 indicate the results of the study. FIG. 9 shows that prior to treatment, seven of the subjects including six male subjects aged 40 to 80 and one female subject of age 70 had an average level of circulating HSP70 of just over 30 μg/ml. Following the treatment, these subjects had an average level of circulating HSP70 of almost 5 μg/ml.

FIG. 10 shows that prior to treatment, three of the subjects including two male subjects aged 45 to 80 and one female subject of age 70 had very low level of detectable circulating HSP70 of 0.1 μg/ml and this HSP70 level remained substantially unchanged following treatment.

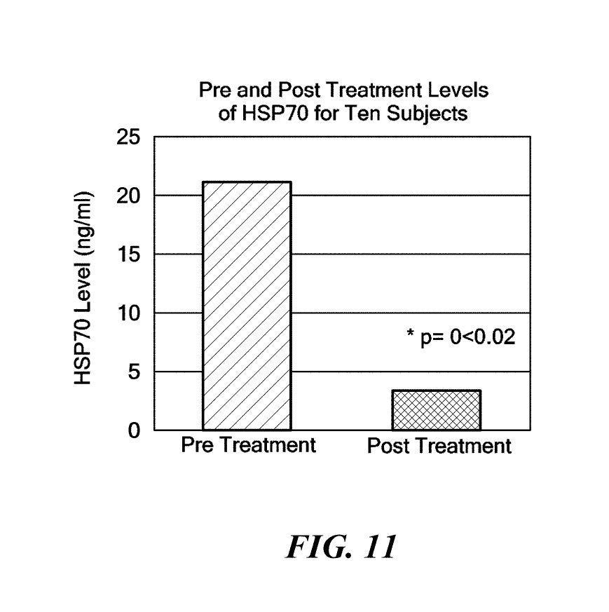

FIG. 11 shows that prior to treatment, the ten subjects including the eight male subjects and the two female subjects had an average level of circulating HSP70 of approximately 21 μg/ml and following treatment, an average level of circulating HSP70 of approximately 3 μg/ml.

It was concluded that treatment with the composition of the invention significantly decreased the level of circulating extracellular HSP70 where extracellular circulating protein was detectable prior to treatment. It has been reported that extracellular HSP70 circulating in the blood stream typically increases with age, Radons 2016, as previously discussed. It can be hypothesized that such increase is due to either the body's reaction to age-induced inflammation and/or HSP70 leakage from cells. FIG. 11 shows that on average, administration of the composition caused an alteration of the chaperone axis such that the typical age-induced level of circulating extracellular HSP70 decreased in subjects post-treatment.

### EXAMPLE 3

The effect of the administration of the composition of the invention on three thoroughbred horses was studied. Each of the thoroughbred horses of the example presented with lameness and a specific ultrasonic lesion in a major ligament or tendon. Prior to treatment, a blood sample was taken from each of the horses and analyzed for a pre-treatment baseline HSP70 content using an Elisa procedure adapted for the analysis of horse plasma. The composition of the invention was prepared by mixing 300 mg Feto-placental Unit which had been harvested and dissected from a ewe including its fetus in 3 ml Emu oil. A dose of the composition was administered to each of the horses using a transcutaneous administration under the axilla at one month intervals for a period of four months. After each monthly treatment, the lesion of each of the horses was monitored via ultra sound.

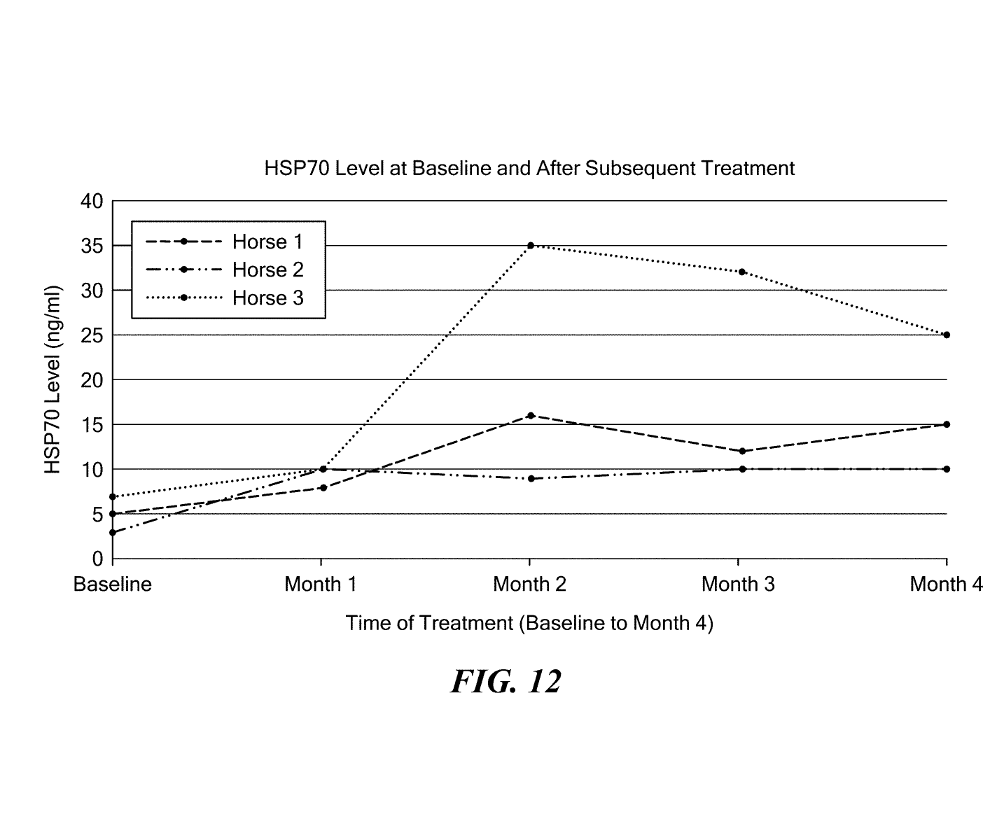

The results of the study are presented in FIG. 12. In all three horses, full resolution of the lesion was achieved. In each horse there was a significant rise in systemic HSP70 associated with treatment.

### EXAMPLE 4

A total of 42 domestic cats including 21 male cats and 21 female cats were provided for study. The cats were selected for the study according to the following criteria:
minimum age of 8 years;
physical health as defined by routine physical examination;

stable body weight within normal season variation for a period of time of at least 6 months prior to the onset of the study;
absence of serious illness which might require euthanasia within 18 weeks of the study; absence of hypothyroidism; and
absence of fractious behavior rendering the subject cat unsuitable for repeated collection of plasma samples.

The selected cats were housed in groups of 8-10 cats in indoor-outdoor purpose colony cages built at the Centre for Feline Nutrition Unit at Massey University, Palmerston North, New Zealand. The cats were fed a complete and balanced commercial high moisture low carbohydrate (canned) feline diet and had ad libitum access to water.

Two cats were deemed too fractious for sampling and were excluded after initial measurement of the GFR. Another cat died during the early part of the trial. The final allocation of 20 male cats and 19 female cats for the study included 10 males and 10 females in the treatment population and 10 males and 9 females in the control population. The oldest and youngest cats studied were respectively 14.93 and 9.31 years of age while the mean age of the studied cats was 11.22 years.

The husbandry of the cats was compiled under the Massey University ethic committed protocol number 12/12. All studies were approved by the Animals Welfare and Ethics committee of Massey University, New Zealand.

The cats were then divided into groups of 10 and the GFR of each cat was measured using the iohexol clearance method. The cats were then separated into a control group and a treatment group to provide a balanced distribution of the measured GFR in each group. Following the separation of the cats into 2 groups, there was no significant difference in the distribution of the measured GFR between the two groups. The trial commenced approximately 6 weeks later, maintaining a staggered approach, with 10 cats starting treatment each week for 4 weeks.

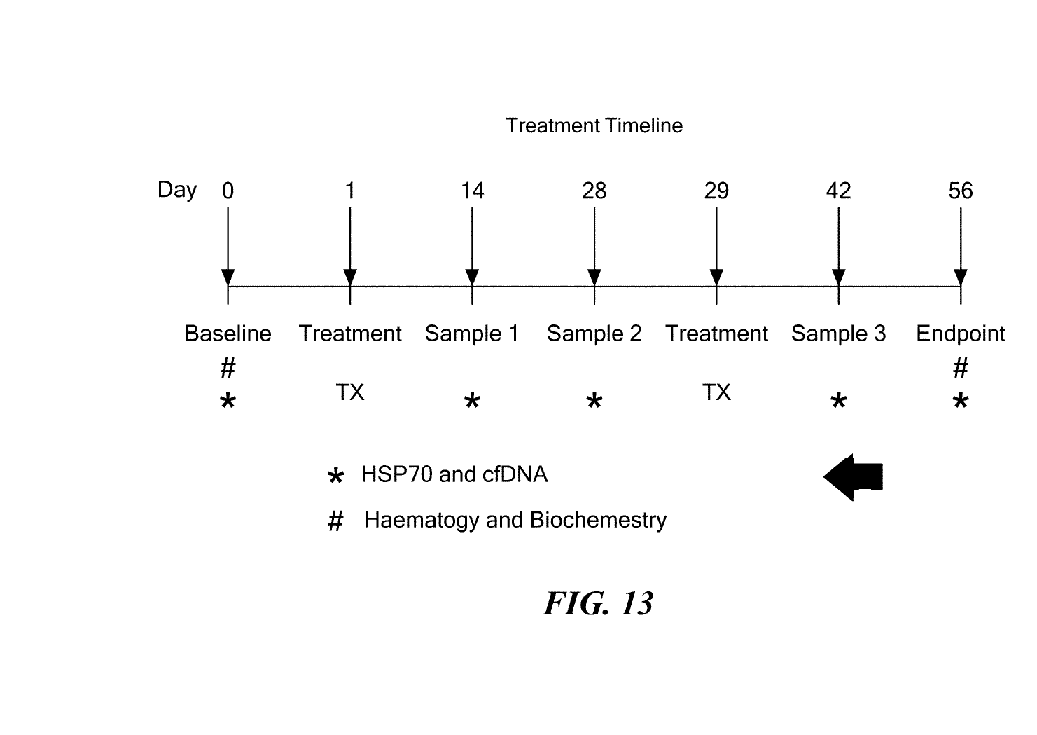

A venous blood sample of each cat was taken on day 1, 14, 28, 42 and 56 of the study, as shown in FIG. 13. Measurements of GFR, Circulating Plasma HSP70 and cell free DNA were taken on days 0 and 56. Each cat was injected with a subcutaneously in the interscapular region with either a treatment or a control saline solution on days 1 and 29. Control and treated cats were paired on the basis of their baseline GFR values. Injection sites were palpated for 1 week after each injection. All cats were weighed weekly during the trial period.

Blood Analysis

All blood samples were collected by jugular venipuncture, after application of 2% lignocaine gel and placed into EDTA tubes for circulating plasma harvesting. On days 0 and 56, a sample of EDTA blood, and clotted blood were submitted to the diagnostic laboratory for routine hematology and biochemistry. Blood placed into plain tubes for serum collection were allowed to clot at room temperature for 15-30 minutes then centrifuged. Within 30 minutes of collection EDTA plasma samples were centrifuged at 1300 rpm in a refrigerated centrifuge and plasma was separated. All serum and plasma samples were stored at −80° C. until transported for assaying.

GFR Measurement

The GFR was determined in each cat using the plasma clearance of iohexol (PCio) method as described by Miyamoto (2001)4. Plasma clearance of iohexol was determined after IV administration of Iohexol and plasma concentrations of iodine were measured by use of a colorimetric assay. Results for PCio were compared with simultaneously obtained values for urinary clearance of creatinine (CCr).

HSP70

Frozen plasma was transported to Endocrinology Labs, School of Veterinary Medicine, UC Davis, USA for HSP70 analysis. The sample dilution was 1:1 dilution, 200 μl buffer mixed with 200 μl serum, followed by the addition of 100 μl of the diluted serum to the wells. ELISA analysis was performed using High sensitivity ELISA Kit (Cat: ADI-EKS-715 Enzolifesciences, Farmingdale, N.Y. 11735), according to the manufacturer's instruction.

Cell Free DNA

The Qubit dsDNA BR (broad range) assay kit was used to determine cell free DNA concentration. The cell free DNA assays were performed using the general assay kits protocol, which utilizes a simple mix and read format for DNA. A number of 0.5 mL tubes were set up for standards and samples. The Qubit™ working solution was prepared by diluting the reagent 1:200 in Qubit™ protein buffer. The Qubit™ working solution was pipetted in an amount of 190 uL of into each of the tubes used for standards. An amount of 10 uL of each Qubit™ standard was added to the appropriate tube and vortex. The Qubit™ working solution was pipetted into individual assay tubes so that the final volume in each tube after adding sample was 200 uL. Each sample was pipetted into a corresponding assay tube and mixed. From the Home Screen of the Qubit® 2.0 Fluorometer, the Protein was selected and a new calibration was run. Upon the completion of the measurement, the result for each sample was read from the screen, removed from the Fluorometer, and the next sample inserted. This procedure was repeated until all the samples were read.

Results

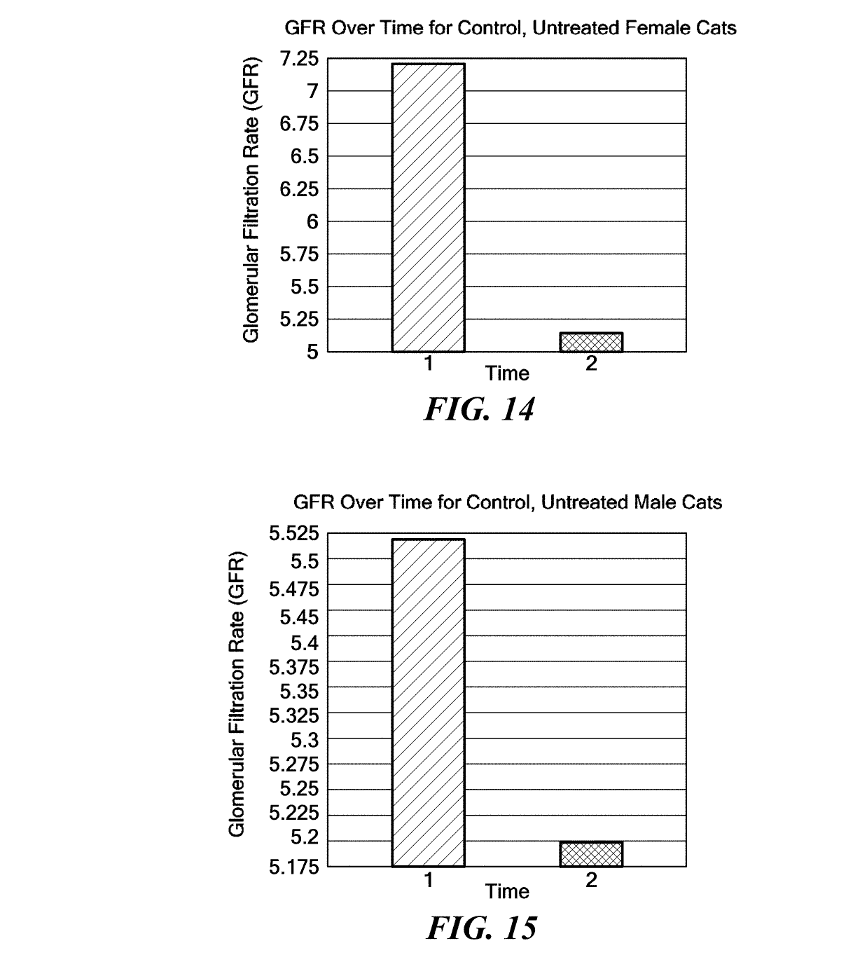

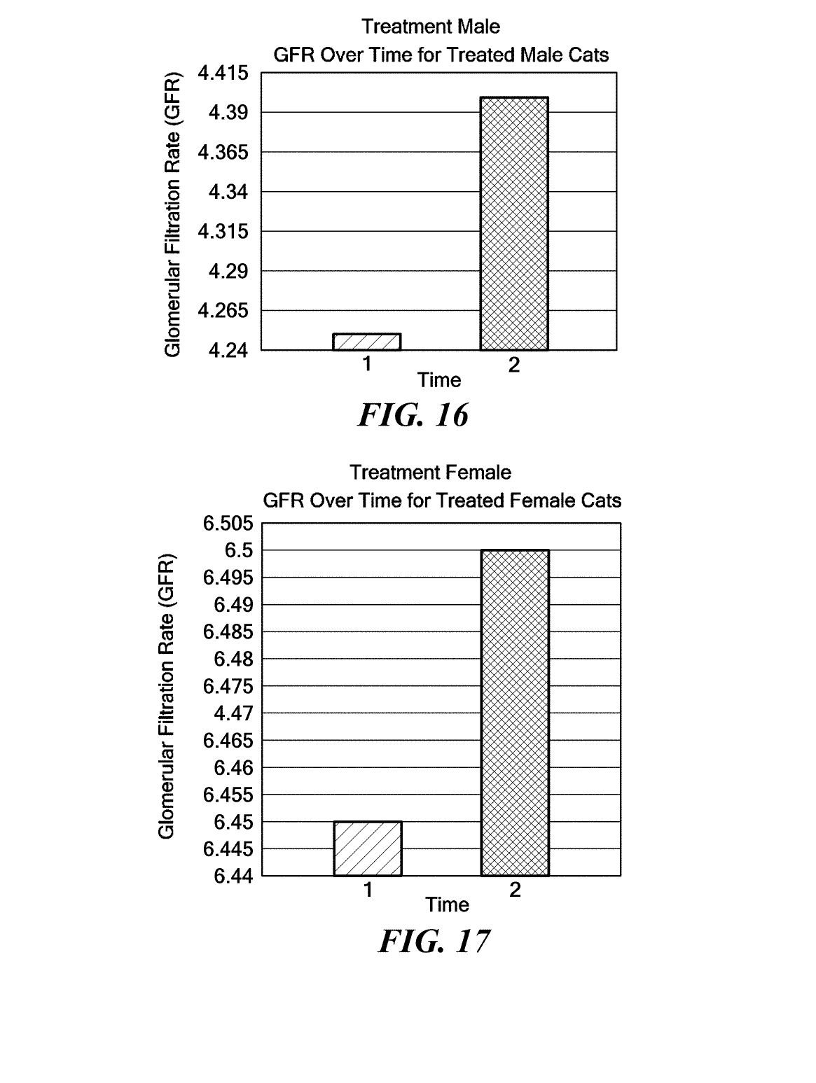

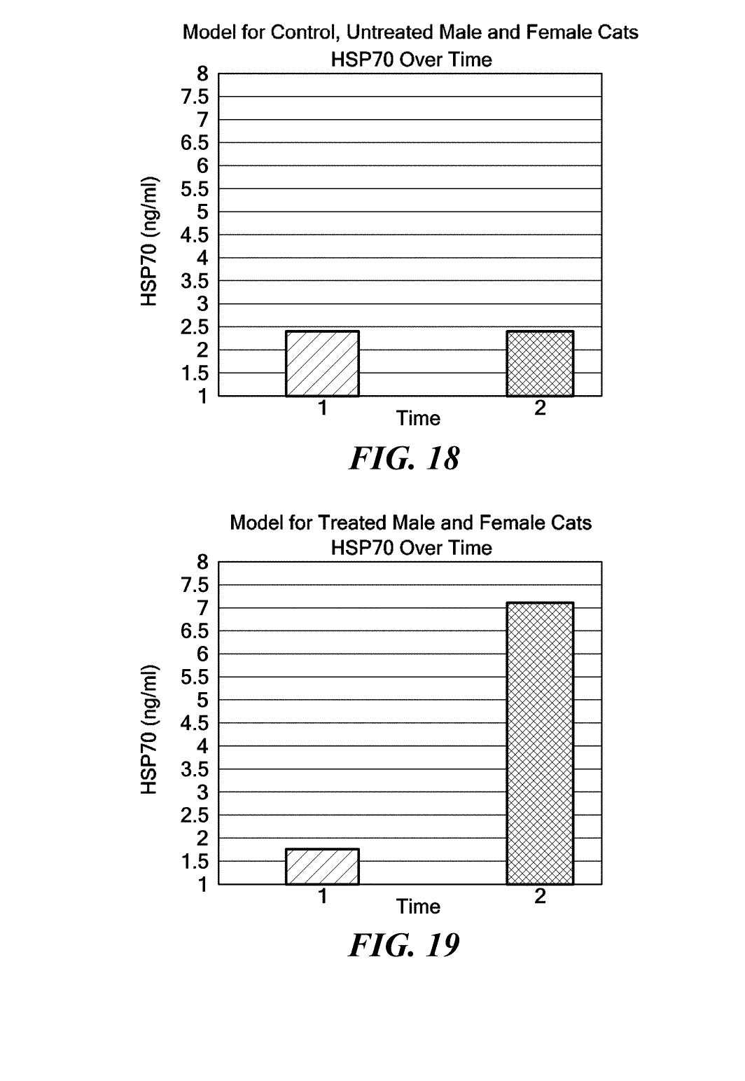

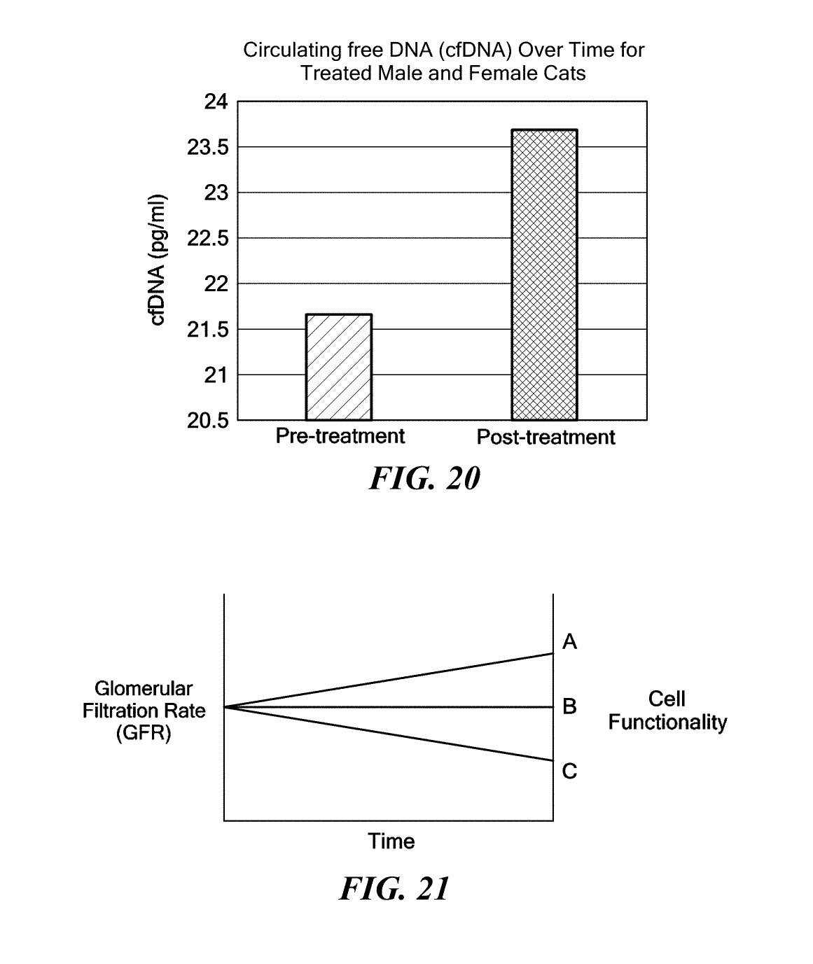

The control non-treated male and female cats showed a decreased GFR at the end of the assessment period, as shown in respective FIGS. 14-15. Treated male and female cats showed increased GFR at the end of the assessment period, as shown in respective FIGS. 16-17. Gender appeared to influence GFR both before and after treatment. FIG. 18 shows that control, untreated male and female cats had a substantially constant level of extracellular HSP70 over time, while the treated male and female cats had an increase in extracellular HSP70 over time and an increase in cell-free DNA, as shown in respective FIGS. 19 and 20. Thus, the administration of the composition to the treated cats resulted in an alteration in the chaperone axis resulting in an increase in extracellular circulating HSP70.

FIG. 21 graphically represents the progression of renal cell/organ function correlated to GFR measurement over time. For example, the natural decrease in GFR over time in the untreated, cats is correlated to a decrease in renal cell/organ function. The decrease in renal cell/organ function represents the normal progression of necrosis/apoptosis in the untreated cats, as shown by line “C” in FIG. 21. Successful treatment can possibly prevent the typical decrease in GFR over time in the treated cats. In such an example, the GFR in the treated cats would remain constant over time. The constant GFR would be correlated to the prevention of the expected decrease in renal cell/organ function, that is, the prevention of the expected progression of necrosis/apoptosis over time, as shown by line “B” in FIG. 21. In the example of the present invention, an increase in GFR over time was measured in the treated cats. Thus, the increase in GFR is correlated to a conversion of non-functional cells from the state of apoptosis to the state of anastasis, as shown by line A of FIG. 21.

### EXAMPLE 5

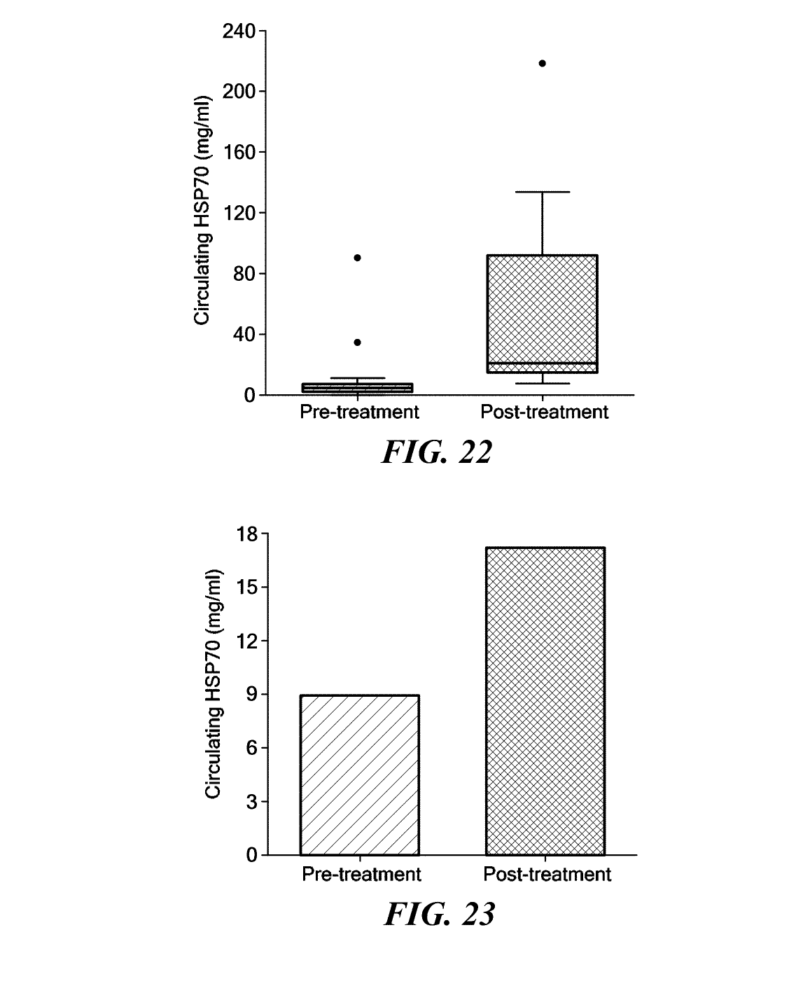

The effect of the administration of the composition on alteration of the CPA axis was studied in eighteen thoroughbred horses. The thoroughbred horses varied in age from 2 to 7 years of age. The thoroughbred horses were all male. Each of the thoroughbred horses presented with some type of stress based musculo-skeletal condition related to their past/present history of horse racing including, for non-limiting examples, bone remodeling, rhabdomyolysis, facture repair, tendon repair, and exercised induced pulmonary hemorrhage. Prior to treatment, a blood sample was taken from each of the horses and analyzed for a pre-treatment baseline HSP70 content using an Elisa procedure adapted for the analysis of horse plasma. The composition of the invention was prepared by mixing in 6 ml sterile saline 300 mg Feto-placental Unit which had been harvested and dissected from a ewe including its fetus. A dose of the composition was administered to each of the horses using an intra-muscular administration on Day 0, A blood sample of each of the horses was then taken on Day 28 and the distribution of the corresponding data is shown in the box plot of FIG. 22.

FIG. 22 shows that the administration of the composition in the thoroughbred horses resulted in an average increase in the level of extracellular HSP70 circulating in the blood stream of the horses. Higher levels of intra-cellular HSP70 proteins have been observed in cancerous cells relative to non-malignant cells, Radons 2016, as previously discussed. It can be hypothesized that a corresponding decrease in extracellular HSP70 proteins circulating in the blood stream can be due to the body's inability to efficiently process and/or release HSP70 from cells as a result of a disease, disorder or condition. FIG. 22 shows that administration of the composition to the horses each exhibiting a stress related condition or disease caused an alteration of the chaperone axis in the horses; HSP70 was more efficiently processed and released by cells into the blood stream of the treated subjects thereby resulting in an increase in circulating extracellular HSP70 post-treatment.

### EXAMPLE 6

The effect of the administration of the composition for CPA axis alteration was studied in a male 88 years of age. At the onset of the study, the subject exhibited signs of ongoing clinical disease including bone marrow suppression leading to a Myelofibrosis blood disorder. The composition of the invention was prepared and administered following the procedures outlined in EXAMPLE 2, and at Day 0 pre-treatment and at Day 35 post-treatment, the level of extracellular HSP70 proteins circulating in the blood stream of the subject was measured by using an Elisa kit for protein detection and quantification.

FIG. 23 shows a pre-treatment level significantly lower than the post-treatment level of extracellular HSP70 circulating in the blood stream of the subject. Higher levels of intra-cellular HSP70 proteins have been observed in cancerous cells relative to non-malignant cells, Radons 2016, as previously discussed. It can be hypothesized that a corresponding decrease in extracellular HSP70 proteins circulating the in blood stream can be due to the body's inability to efficiently process and/or release HSP70 from cells as a result of disease. FIG. 23 shows that administration of the composition to an individual suffering clinical disease caused an alteration of the chaperone axis; HSP70 was more efficiently processed and released by cells into the blood stream thereby resulting in a increase in post-treatment circulating extracellular HSP70.

The foregoing examples and description are not to be deemed limiting of the invention which is defined in the following claims. The invention is understood to encompass such obvious modifications thereof as would be apparent to those of ordinary skill in the art.

What is claimed is:

1. A method of treatment of a subject comprising:
administering to the subject at least one dose of a therapeutically effective amount of a composition for altering a chaperone protein axis of the subject;
wherein the composition includes at least one essential fragment of a feto-placental unit protein.

2. The method of claim 1, wherein the essential fragment of the feto-placental unit protein comprises an essential fragment of a chaperone protein.

3. The method of claim 2, wherein the chaperone protein comprises a protein included in the HSP70 family.

4. The method of claim 1, wherein prior to the administering step, the method comprises identifying a subject in need of alteration of the chaperone protein axis of the subject.

5. The method of claim 1, wherein altering the chaperone protein axis of the subject comprises stimulating the chaperone protein axis for at least one of manufacturing a chaperone protein, releasing a chaperone protein from a cell, decreasing an amount of a chaperone protein, and increasing an amount of a chaperone protein in the subject.

6. The method of claim 1, wherein altering the chaperone protein axis of the subject comprises decreasing an amount of a chaperone protein circulating in a blood stream of the subject.

7. The method of claim 1 further comprising monitoring the chaperone protein axis in the subject during at least one of a selected pre-treatment time period, a treatment time period, and a post-treatment time period.

8. The method of claim 7, wherein the monitoring comprises measuring at least one of an intracellular amount and an extracellular amount of a key protein which is involved in the chaperone protein axis in the subject.

9. The method of claim 8, wherein the key protein comprises a protein included in the HSP70 family.

10. The method of claim 1, wherein altering the chaperone protein axis comprises at least one of growing a cell, regenerating a cell and repairing a cell in the subject.

11. The method of claim 1, wherein the therapeutically effective amount is in a range of 3 mg of the composition per 100 kg body weight of the subject to 3 g of the composition per 100 kg body weight of the subject.

12. The method of claim 1, wherein administering comprises at least one administration procedure selected from a group consisting of an oral administration, a rectal administration, an inhalation administration, a cutaneous administration, a subcutaneous administration, a transcutaneous administration, an intravenous injection, and an intramuscular injection.

13. The method of claim 1, wherein administering comprises administering the at least one dose of the therapeutically effective amount of the composition in combination with at least one additional component for achieving an additive or synergistic effect.

14. The method of claim 13, wherein administering the at least one dose of the therapeutically effective amount of the composition in combination with at least one additional component for achieving an additive or synergistic effect comprises a transcutaneous administration.

15. The method of claim 14, wherein the additional component is an oil.

16. The method of claim 15, wherein the oil is an emu oil.

17. The method of claim 1, wherein administering comprises administering the at least one dose at a selected dosage rate over a selected dosing time interval.

18. The method of claim 17, wherein the selected dosage rate comprises a range of one dose per one day to one dose per 365 days during a selected dosing time interval.

19. The method of claim 18, wherein the selected dosing time interval comprises a range of a single day to a day substantially proximate to an expected end of life of the subject.

20. The method of claim 1, wherein altering the chaperone protein axis of the subject comprises altering a level of the chaperone protein axis from a pre-alteration chaperone protein axis level to a post-alteration chaperone protein axis level which is maintained for a sustaining period of time.

21. The method of claim 20, wherein the sustaining period of time is in a range of 1 week to 52 weeks.

22. The method of claim 20, wherein the post-alteration chaperone protein axis level is maintained for a sustaining period of time after the at least one dose is no longer being administered to the subject.

23. The method of claim 1, wherein the subject is a mammal.

24. The method of claim 23, wherein the mammal is a human.

25. The method of claim 24, wherein the mammal is a horse.

26. A pharmaceutical composition comprising:
a therapeutically effective amount of at least an essential fragment of a feto-placental unit protein for altering a chaperone protein axis in a subject.

27. A kit containing the pharmaceutical composition of claim 26.

28. The pharmaceutical composition of claim 26,
wherein in a subject which is human, the therapeutically effective amount is in a range from about 10 ug feto-placental unit protein/kg body weight to 1 mg feto-placental unit protein/kg body weight in the subject; and
wherein in a subject which is a horse, the therapeutically effective amount is in a range from about 1 ug feto-placental unit protein/kg body weight to 1 mg feto-placental unit protein/kg body weight in the subject.

29. A method of assessing an ability of a subject to recover from a condition related to a defect, disease, injury or other trauma including the steps of:
administering at least one dose of an effective amount of a composition to a subject for altering a chaperone protein axis in the subject;
wherein the composition includes at least an essential fragment of a feto-placental unit protein;
measuring at least one pre-alteration amount of at least one key protein of the chaperone protein axis at at least one selected time interval before administering the at least one dose;
measuring at least one post-alteration amount of the at least one key protein of the chaperone protein axis at at least one selected time interval following administering the at least one dose; and
correlating with a first algorithm the difference in the at least one pre-alteration amount and the at least one post-alteration amount of the at least one key protein with an ability of the subject to recover from the condition.

30. The method of claim 29, wherein the condition is selected from the group consisting of a tendon injury, a muscle injury and at least one aging condition.

31. The method of claim 29, wherein the essential fragment of the feto-placental unit protein comprises an essential fragment of a chaperone protein.

32. The method of claim 31, wherein the chaperone protein comprises a protein included in the HSP70 family.

33. The method of claim 29, wherein the key protein comprises a protein included in the HSP70 family.

34. The method of claim 29, wherein the effective amount is in a range of 1 ug composition to 10 g composition per 50 kg body weight of the subject.

35. The method of claim 29, wherein administering the composition comprises at least one of a plurality of administration procedures selected from an oral administration, a rectal administration, an inhalation administration, a cutaneous administration, a subcutaneous administration, a transcutaneous administration, an intravenous injection, and an intramuscular injection.

36. The method of claim 29, wherein administering the composition comprises administering transcutaneously the at least one dose in combination with at least one additional component for achieving an additive or synergistic effect.

37. The method of claim 29, wherein administering the administering the at least one dose comprises a transcutaneous administration of the composition in combination with at least one additional component for achieving an additive or synergistic effect.

38. The method of claim 37, wherein the additional component is selected from the group consisting of an oil.

39. The method of claim 38, wherein the oil is an emu oil.

40. The method of claim 29, wherein administering the at least one dose comprises the at least one dose at a selected dosage rate over a selected dosing time interval.

41. The method of claim 40, wherein the selected dosage rate is in a range of one dose per one day to one dose per 365 days during a selected dosing time interval.

42. The method of claim 40, wherein the selected dosing time interval is in a range of a single day to a day substantially proximate to an expected end of life of the subject.

43. The method of claim 29, wherein the subject is a mammal.

44. The method of claim 29, wherein the subject is a horse.

45. The method of claim 29, wherein the subject is a human.

46. A kit for assessing an ability of a subject to recover from a condition related to a disease, injury or other trauma comprising:
a composition for altering a chaperone protein axis of the subject;
wherein the composition includes at least an essential fragment of a feto-placental unit protein; and
a device for administering the composition.

47. The kit of claim 46 further comprising one or more devices for measuring at least one pre-alteration amount of a key protein of the chaperone protein axis at at least one selected time interval before administering the composition and at least one post-alteration amount of the key protein of the chaperone protein axis at at least one selected time interval following administering the composition.

48. The kit of claim 47 further comprising at least one device for correlating with a first algorithm the difference in the at least one pre-alteration amount and the at least one post-alteration amount of the key protein with an ability of the subject to recover from the condition.
